(19) United States
(12) Patent Application Publication
(10) Pub. No.: US 2025/0345057 A1
(43) Pub. Date: Nov. 13, 2025

(54) SURGICAL LAYER FIXATION DEVICE

(71) Applicant: Mesh Fastening LLC, Atlanta, GA (US)

(72) Inventors: Christopher Robert Kuzniak, Atlanta, GA (US); Courtney Okerblad, Atlanta, GA (US); John Tipton, Atlanta, GA (US); Martin Conrad Jacobson, Atlanta, GA (US); Franco Zapata, Atlanta, GA (US); Olivia Brencovich, Atlanta, GA (US)

(21) Appl. No.: 19/202,176

(22) Filed: May 8, 2025

Related U.S. Application Data
(60) Provisional application No. 63/644,486, filed on May 8, 2024.

(51) Int. Cl. A61B 17/064; A61B 17/00; A61B 17/072

(52) U.S. Cl. CPC A61B 17/064; A61B 17/07207; A61B 2017/00367; A61B 2017/0647; A61B 2017/0649; A61B 2017/07271; A61B 2017/07278; A61B 2017/07285

(57) ABSTRACT

A surgical tacking system and method for fixation of soft tissues or soft prostheses during surgical procedures, such as ventral hernia repairs, e.g., between two tissues or between the tissue and the implant. The tacking system operates on a two-part fixator consisting of a penetrating tack and a retention collar to quickly and reliably deploy and fixate tissue in a closing procedure. The fastener dispenser includes a first jaw movably coupled to a second jaw that dispenses a fastener. The fastener dispenser is movable from an unclamped configuration to a clamped configuration, thereby reducing a relative spacing between the first jaw and the second jaw so as to hold the first layer relative to the second layer therebetween, while penetrating the first layer and the second layer with the elongated member to couple the elongated member to the collar to fixate the first layer to the second layer.

### CROSS-REFERENCE TO RELATED APPLICATIONS

This application claims priority to and the benefit of U.S. Provisional Patent Application No. 63/644,486, filed on May 8, 2024, the disclosure of which is expressly incorporated herein by reference in its entirety.

### BACKGROUND

Hernias involve the bulging of an organ or part of an organ out of the body cavity that typically contains it. In particular, ventral hernia is a common condition that often requires surgical intervention. The surgical repair of ventral hernias is often performed in conjunction with implants such as meshes, which are designed to strengthen the repair and reduce the risk of hernia recurrence.

The current standard of care for ventral hernia repair typically involves a surgical intervention to fix meshes to the anatomy at the defect site. Specifically, the hernia is closed by accessing the site through an open or minimally invasive approach and placing a mesh (i.e., a surgical mesh, mesh implant, or mesh prosthesis) to reinforce the repaired defect. Placement and fixation of the surgical mesh may be anterior to the repaired fascia and abdominal wall muscles (i.e., onlay repair) posterior to the abdominal wall muscles (i.e., underlay repair), or posterior to the abdominal wall muscles (i.e. underlay repair) to reinforce the repaired defect. A typical technique for underlay repair entails placement of the surgical mesh in the retro-rectus position after closure of the posterior fascia, which is subsequently secured to the anterior fascia. However, underlay repair may instead involve placement of a mesh posterior to the abdominal muscles but anterior the peritoneum. When the fascial defect cannot readily be closed, a bridging mesh may be placed in line with the fascial defect (i.e., inlay repair). The mesh is fixated in place by suturing, tacking, or gluing the perimeter and the abdominal wall is then closed.

Attachment (i.e., fixing or securing) of mesh prostheses during the surgical repair of large ventral and incisional hernias is a time-consuming process. Studies conducted within the past few years report the average procedure time for open onlay and underlay repairs range from 60 to 80 minutes. Furthermore, long overall operating times are associated with mesh infection, which can necessitate reoperation. Therefore, a need exists for faster and more secure surgical mesh fixation to reduce overall operating time in hernia repairs.

### SUMMARY

An exemplary surgical tacking system and method are disclosed for fixation of soft tissues or soft prostheses during surgical procedures, such as ventral hernia repairs. The fixation can be between two tissues (as attachments) or between the two tissues and the implant (e.g., mesh prostheses). The tacking system operates on a two-part fixator consisting of a penetrating tack and a retention collar to quickly and reliably deploy and fixate tissue in a closing procedure.

The tack preferably features a high-pitch spiral to aid in tissue penetration and a series of biased ribs to allow for variable thickness placements. Once the tack is placed through relevant layers, the retention disk snaps down over the tack and can be indexed at several points along the tack length to generate optimal pressure on the tissues. Tacks and disks are separately held in the upper and lower jaws of the device and are stowed as a cartridge. When the trigger is pulled, the first stage of the stroke grips the tissues, and the second stage delivers the tack and locks on the retention disk, securing tissues there between. The tack can be dispensed in an inward direction toward the patient or an outward direction away from the patient.

The exemplary system can be employed as a single-use disposable device, for a given operation that can loaded with two cartridges and reloaded via replacement cartridges, comprising the two-part fixators. The exemplary system is contemplated to improve the onlay and sublay repairs, e.g., for hernia, by 25%-50%. In addition, the exemplary system is contemplated to improve operating room safety in replacing surgical needles that are potential hazards to the surgeon and technicians with fully-enclosed fixation components.

In some aspects, the techniques described herein relate to a surgical fastening system for fixing a first layer to a second layer. The system includes a fastener dispenser including a first jaw and a second jaw. The first jaw includes a proximal end and a distal end and defines a first jaw track therein extending between the proximal end and the distal end. The second jaw includes a proximal end and a distal end and defines a second jaw track therein extending between the proximal end and the distal end. The system also includes a fastener having an elongated member and a collar. The elongated member spans the combined thickness of the first layer and the second layer and is stored within the second jaw track. The collar is stored within the first jaw track. The first jaw is movably coupled to the second jaw so that the fastener dispenser is movable from an unclamped configuration, in which the first jaw and the second jaw have a relative spacing on opposite sides of the first layer and the second layer, to a clamped configuration, in which the second jaw is moved relative to the first jaw to reduce the relative spacing between the first jaw and the second jaw. By moving from the unclamped configuration to the clamped configuration, the fastener dispenser is able to hold the first layer relative to the second layer.

In various implementations, one of the first or second jaws further includes an ejection jaw movably coupled to the one of the first or second jaws. The ejection jaw includes a protrusion configured to extend toward the track of the one of the first or second jaws to which the ejection jaw is coupled. Moving the ejection jaw toward the one of the first or second jaws engages the protrusion against one of the elongated members or the collar stored in the track of the one of the first or second jaws, thereby urging the one of the elongated member or the collar toward the other of the elongated member or the collar to couple the elongated member and the collar.

In various implementations, an ejection jaw biasing member urges the ejection jaw toward the one of the first or second jaws to which the ejection jaw is coupled.

In various implementations, the elongated member extends between a base end and a penetrating end and defines an elongated member central axis.

In various implementations, the elongated member is stored within the second jaw track such that the elongated member central axis is parallel with or forms an acute angle with an extension axis of the second jaw track.

In various implementations, the second jaw further includes a pivoting feature configured to pivot the elongated member when the elongated member is dispensed such that the penetrating end of the elongated member extends toward the collar.

In various implementations, the system further includes a cutting blade disposed adjacent to the first jaw, the cutting blade configured to remove a portion of the penetrating end of the elongated member on a side of the collar opposite the base end of the elongated member after the elongated member is coupled to the collar.

In various implementations, the system further includes a collar cartridge configured to store and dispense the collar. In various implementations, the collar is one of a plurality of collars stored in the collar cartridge. In various implementations, the system further includes an elongated member cartridge configured to store and dispense the elongated member. In various implementations, the elongated member is one of a plurality of elongated members stored in the elongated member cartridge. In various implementations, the first jaw track is configured to receive the collar cartridge, and the second jaw track is configured to receive the elongated member cartridge. In various implementations, a first biasing member urges the collar cartridge toward the distal end of the first jaw. In various implementations, a second biasing member urges the elongated member cartridge toward the distal end of the second jaw.

In various implementations, the elongated member is formed integrally with the second jaw and extends toward the first jaw, wherein the system further includes a dispensing blade configured to remove the elongated member from the second jaw.

In various implementations, the system further includes a housing, the housing including a first actuator movable between a first unengaged position and a first engaged position, wherein movement between the first unengaged position and the first engaged position moves the fastener dispenser between the unclamped configuration to the clamped configuration, respectively. In various implementations, the housing further includes a second actuator movable between a second unengaged position and a second engaged position, wherein movement between the second unengaged position and the second engaged position moves the ejection jaw relative to one of the first or second jaws to urge one of the elongated member or the collar toward the other of the elongated member or the collar to couple the elongated member and the collar.

In various implementations, the system further includes a handle configured to move the first actuator between the first unengaged position and the first engaged position. In various implementations, the handle is further configured to move the second actuator between the second unengaged position and the second engaged position.

In various implementations, the system further includes a hollow shaft extending from the housing to the fastener dispenser. In various implementations, the first jaw is rotationally fixed to the hollow shaft. In various implementations, the fastener dispenser is detachably coupled to the hollow shaft for disposal or replacement.

In various implementations, the first layer is a surgical mesh implant, and the second layer is a patient's tissue. In various implementations, the surgical mesh implant is a hernia mesh. In various implementations, both the first layer and the second layer are a patient's tissue.

In some aspects, the techniques described herein relate to a surgical fastener for fixing a first layer to a second layer. The fastener includes an elongated member extending between a base end and a penetrating end and defining an elongated member central axis and a collar defining an opening configured to receive the penetrating end of the elongated member. The elongated member spans the combined thickness of the first layer and the second layer. The elongated member is configured to engage with the opening of the collar, thereby coupling the elongated member and the collar. The fastener operates in a system that includes a fastener dispenser, the fastener dispenser including: a first jaw including a proximal end and a distal end, the first jaw defining a first jaw track therein extending between the proximal end and the distal end; and a second jaw including a proximal end and a distal end, the second jaw defining a second jaw track therein extending between the proximal end and the distal end. The proximal end of the first jaw is pivotably coupled to the proximal end of the second jaw. The elongated member is stored within the second jaw track, and the collar is stored within the first jaw track.

In various implementations, the elongated member includes a radially extending engagement feature. In various implementations, the radially extending engagement feature of the elongated member further includes a radially outwardly extending rib. In various implementations, the opening of the collar further includes a radially extending engagement feature configured to couple with the radially extending engagement feature of the elongated member. In various implementations, the engagement feature of the elongated member further includes a radially outwardly extending rib, wherein the engagement feature of the collar defines a groove, and wherein the radially outwardly extending rib of the elongated member couples with the groove of the collar.

In some aspects, the techniques described herein relate to a method for surgically fixing a first layer to a second layer. The method includes: (i) providing a fastener dispenser including: a first jaw having a proximal end and a distal end, the first jaw defining a first jaw track therein extending between the proximal end and the distal end; a second jaw having a proximal end and a distal end, the second jaw defining a second jaw track therein extending between the proximal end and the distal end; (ii) providing a fastener including: an elongated member and a collar, the elongated member spanning the combined thickness of the first layer and the second layer, wherein the elongated member extends between a base end and a penetrating end and defines an elongated member central axis; wherein the collar defines an opening configured to receive the penetrating end of the elongated member; and wherein the elongated member is stored within the second jaw track and the collar is stored within the first jaw track; (iii) positioning the fastener dispenser so that the first layer and the second layer are disposed between the first jaw and the second jaw; (iv) moving the fastener dispenser from an unclamped configuration, in which the first jaw and the second jaw have a relative spacing on opposite sides of the first layer and the second layer, to a clamped configuration, in which the second jaw is pivoted relative to the first jaw to reduce the relative spacing between the first jaw and the second jaw, thereby holding the first layer relative to the second layer; and (iv) coupling the elongated member with the collar, thereby clamping the first layer and the second layer between the base end of the elongated member and the collar, thereby fixing the first layer relative to the second layer. In various implementations, at least one of the first layer and second layer are a patient's tissue.

In various implementations, the method further includes: providing an ejection jaw movably coupled to one of the first or second jaws, the ejection jaw including a protrusion configured to extend toward the track of the one of the first or second jaws to which the ejection jaw is coupled.

In various implementations, coupling the elongated member with the collar further includes moving the ejection jaw toward the one of the first or second jaws to which it is coupled engaging the protrusion against one of the elongated members or the collar stored in the track of the one of the first or second jaws, thereby urging the one of the elongated member or the collar toward the other of the elongated member or the collar to couple the elongated member and the collar.

In various implementations, coupling the elongated member with the collar further includes urging the penetrating end of the elongated member through the first layer and the second layer toward the collar and engaging the elongated member with the opening of the collar.

In various implementations, the method further includes pivoting the elongated member from a storage orientation to a dispensing orientation, wherein in the storage orientation, the elongated member is stored within the second jaw track such that the elongated member central axis is parallel with or forms an acute angle with an extension axis of the second jaw track, and wherein in the dispensing orientation, the elongated member central axis is aligned with an opening of the collar.

In various implementations, the first layer is a surgical mesh implant, and the second layer is a patient's tissue.

In various implementations, the surgical mesh implant is a hernia mesh. In various implementations, both the first layer and the second layer are a patient's tissue.

In some aspects, the techniques described herein relate to a surgical fastener for fixing a first layer to a second layer. The fastener includes: an elongated member extending between a base end and a penetrating end and defining a fastener lumen therein, wherein the fastener defines a plurality of openings extending through the elongated member into the fastener lumen, and wherein the fastener defines a longitudinal axis; and a plurality of anchoring members. Each anchoring member includes a proximal end and a distal end, wherein respective proximal ends of each anchoring members are configured to translate axially toward the penetrating end of the fastener, and wherein axial translation of the respective proximal ends of plurality of anchoring members toward the penetrating end of the fastener causes the anchoring members to move from an undeployed configuration, in which the plurality of anchoring members are disposed entirely within the fastener lumen, to a deployed configuration, in which at least a portion of each anchoring member to move radially outward and extend through a respective opening of the plurality of openings.

In various implementations, in the deployed configuration, the portion of the respective anchoring member that extends through the respective opening extends at an angle less than or equal to 90 degrees relative to the longitudinal axis of the fastener.

In various implementations, the plurality of openings are arranged circumferentially around the elongated body adjacent the penetrating end.

In various implementations, in the undeployed configuration, the plurality of anchoring members extend parallel to the longitudinal axis of the fastener.

In various implementations, in the deployed configuration, the portion of the respective anchoring member that extends through the respective opening includes the distal end of the anchoring member.

In various implementations, a plurality of shelves are disposed on an inner surface of the fastener lumen, wherein each respective shelf is disposed adjacent a respective opening of the plurality of openings.

In various implementations, a plurality of shelves extend radially outward from the longitudinal body, wherein each respective shelf is disposed adjacent a respective opening of the plurality of openings.

In various implementations, the distal end of each respective anchoring member is fixed to an inner surface of the fastener lumen at the penetrating end of the fastener.

In various implementations, in the deployed configuration, the portion of the respective anchoring member that extends through the respective opening includes a section of the respective anchoring member disposed between the distal end of the anchoring member and the proximal end of the anchoring member.

In some aspects, the techniques described herein relate to a method for surgically fixing a first layer to a second layer. The method includes: (i) providing a fastener including: an elongated member extending between a base end and a penetrating end and defining a fastener lumen therein, wherein the fastener defines a plurality of openings extending through the elongated member into the fastener lumen, and wherein the faster defines a longitudinal axis; and a plurality of anchoring members. Each anchoring member includes a proximal end and a distal end. The method further includes: (ii) disposing the fastener within a central lumen of a delivery cannula; (iii) advancing the penetrating end of the fastener through the first layer and through the second layer such that the base end of the fastener abuts the second layer; and (iv) moving respective proximal ends of each anchoring members axially toward the penetrating end of the fastener, thereby moving the anchoring members from an undeployed configuration, in which the plurality of flexile anchoring members are disposed entirely within the fastener lumen, to a deployed configuration, in which at least a portion of each anchoring member to move radially outward through a respective opening, thereby fixing the first layer relative to the second layer.

In various implementations, advancing the penetrating end of the fastener through the first layer and through the second layer such that the base end of the fastener abuts the second layer includes applying a distally-directed force onto the base of the fastener via a longitudinally extending tubular fastener driver, wherein the fastener driver defines a central lumen.

In various implementations, moving the respective proximal ends of each anchoring members axially toward the penetrating end of the fastener includes applying a distally-directed force onto the respective proximal ends of the anchoring member via a longitudinally extending anchor driver, wherein the anchor driver is disposed within the fastener driver.

In various implementations, at least one of the first layer and second layer are a patient's tissue.

In various implementations, the first layer is a surgical mesh implant, and the second layer is a patient's tissue.

In various implementations, the surgical mesh implant is a hernia mesh.

In various implementations, both the first layer and the second layer are a patient's tissue.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIGS. 1A-1C show various views of an exemplary fastener dispenser for securing a first layer to second layer, in accordance with an illustrative embodiment. FIG. 1A shows a perspective view of the exemplary fastener dispenser. FIG. 1B shows a side view of the exemplary fastener dispenser of FIG. 1A. FIG. 1C shows a side detail view of the exemplary fastener dispenser of FIG. 1A.

FIG. 2A-2B show exemplary fasteners, in accordance with various illustrative embodiments.

FIGS. 3A-3B show side and top views of an elongated member of a fastener, in accordance with various illustrative embodiments.

FIGS. 4A-4B show side and top views of a collar of a fastener, in accordance with various illustrative embodiments.

FIG. 5A-FIG. 5E shows side partial cross-sectional views of exemplary fastener dispensers, elongated members, and collars according to various illustrative embodiments, wherein the exemplary fastener dispensers include various exemplary biasing mechanisms for advancing the elongated members and collars along the second jaw and first jaw, respectively.

FIG. 6 shows a side partial cross-sectional detail view of the exemplary fastener dispenser of FIGS. 1A-1C, in accordance with an illustrative embodiment.

FIG. 7 shows a side view of an exemplary fastener dispenser coupling an exemplary elongated member and an exemplary collar to form a coupled fastener, thereby securing a surgical mesh to a patient's abdominal wall, in accordance with an illustrative embodiment.

FIG. 8A-8D show various views of an exemplary fastener dispenser, in accordance with an illustrative embodiment.

FIG. 9 shows a side partial cross-sectional view of an exemplary fastener dispenser, in accordance with an illustrative embodiment.

FIGS. 10 shows an exemplary fastener dispenser, in accordance with an illustrative embodiment.

FIG. 11 shows perspective views of first, second, and ejection jaws of a fastener dispenser, in accordance with an illustrative embodiment.

FIG. 12 shows a side view of an elongated member of a fastener and a top view of a collar of a fastener, in accordance with an illustrative embodiment.

FIG. 13 shows a side-perspective view of an exemplary fastener, wherein an elongated member is coupled with a collar, in accordance with an illustrative embodiment.

FIG. 14 shows a side-perspective view of an exemplary fastener, wherein an elongated member is coupled with a collar, in accordance with an illustrative embodiment.

FIG. 15 shows various exemplary elongated members and collars, according to various illustrative embodiments.

FIG. 16 shows various exemplary elongated members according to various illustrative embodiments.

FIGS. 17A-17C show perspective views of an exemplary fastener, wherein FIG. 17A shows an exemplary elongated member, FIG. 17B shows an exemplary collar, and FIG. 17C shows the elongated member of FIG. 17A coupled with the collar of FIG. 17B, in accordance with an illustrative embodiment. FIG. 17D shows a side view of an exemplary fastener dispenser loaded with the exemplary elongated member and collar of FIGS. 17A-17C, in accordance with an illustrative embodiment.

FIGS. 18A-18B show side views of an exemplary fastener for securing a first layer to second layer, in accordance with an illustrative embodiment. FIG. 18A shows the fastener with a plurality of anchoring members in an undeployed configuration. FIG. 18B shows the fastener and flexible anchoring members in a deployed configuration.

FIG. 19 shows a perspective view of an exemplary fastener for securing a first layer to second layer, in accordance with an illustrative embodiment and a fastener delivery system, wherein the plurality of anchoring members is in the deployed configuration.

FIGS. 20A-20C show the exemplary fastener of FIG. 18A-18B in use in an exemplary fastener delivery system. FIG. 20A shows the exemplary fastener of FIG. 18A in the exemplary fastener delivery system with the plurality of anchoring members in the undeployed configuration. FIG. 20B shows the exemplary fastener delivery system moving the exemplary fastener into the first and second layers. FIG. 20C shows the exemplary fastener delivery system moving the plurality of anchoring members to the deployed configuration.

FIGS. 21A-21C show side perspective views of an exemplary fastener for securing a first layer to second layer, in accordance with an illustrative embodiment, and a fastener delivery system. FIG. 21A shows the fastener within the fastener delivery system with the plurality of anchoring members in the undeployed configuration. FIG. 21B shows the fastener moved into the first and second layers with the proximal ends of the plurality of anchoring members angled radially inward. FIG. 21C shows the plurality of anchoring members in the deployed configuration.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

### Example System

In one aspect, provided herein is a surgical fastening system for fixing a first layer (e.g., an implant) to a second layer (e.g., a “target tissue” such as a hernia defect site in a patient). In some implementations, the surgical fastening system includes a fastener dispenser and a fastener. As used herein, an implant may include a soft implant, such as a surgical mesh. Throughout this disclosure, surgical meshes may also be referred to as mesh implants, mesh prostheses, or repair meshes. As used herein, these terms are interchangeable. Furthermore, in the interest of brevity, this disclosure often refers to the first layer as an implant and to the second layer as a patient's tissue. However, this disclosure also contemplates use of the surgical fastening system to fix other layers to one another. For example, the surgical fastening system may be used to fix one layer of tissue to another layer of tissue, fix one layer of an implant to another layer of another implant, or fix one layer of an implant to another layer of the same implant.

### Example Fastener Dispenser

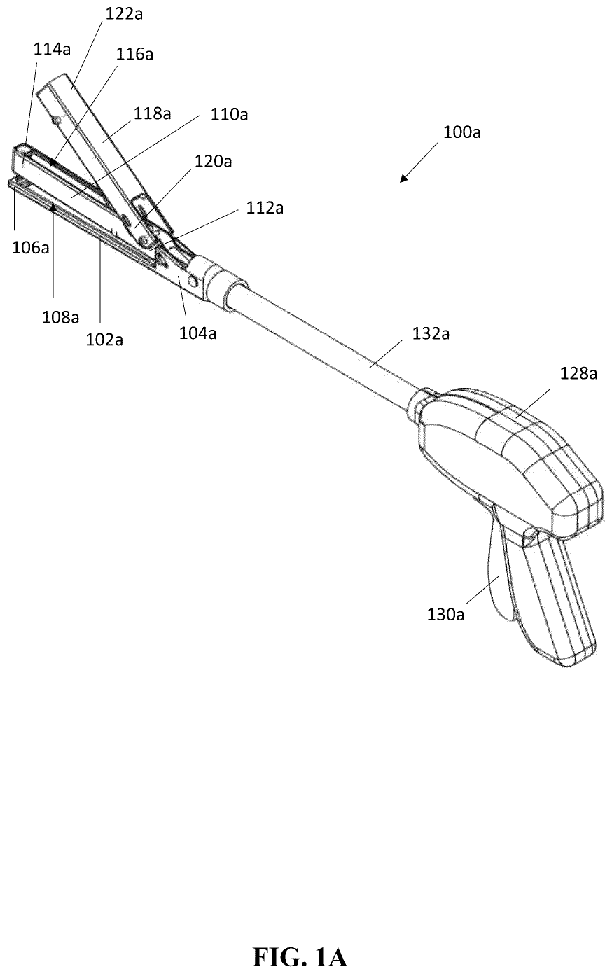

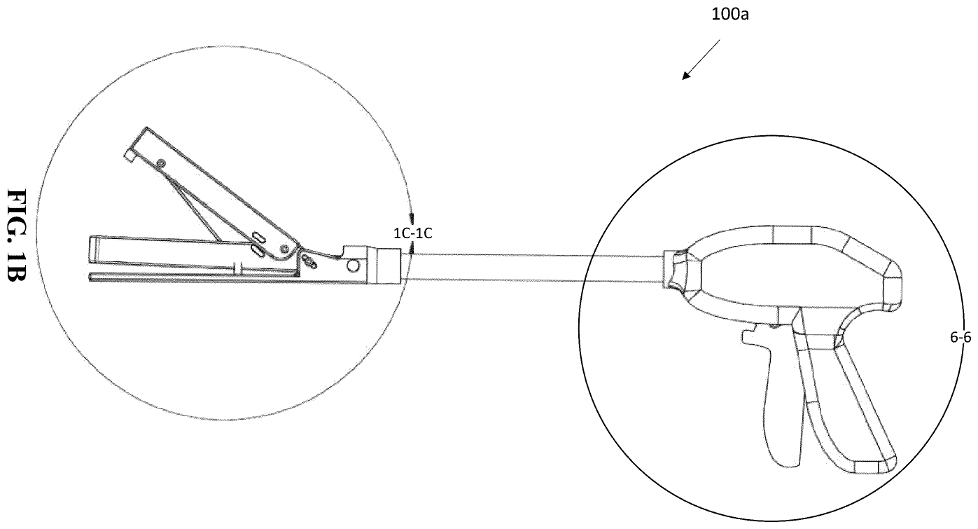

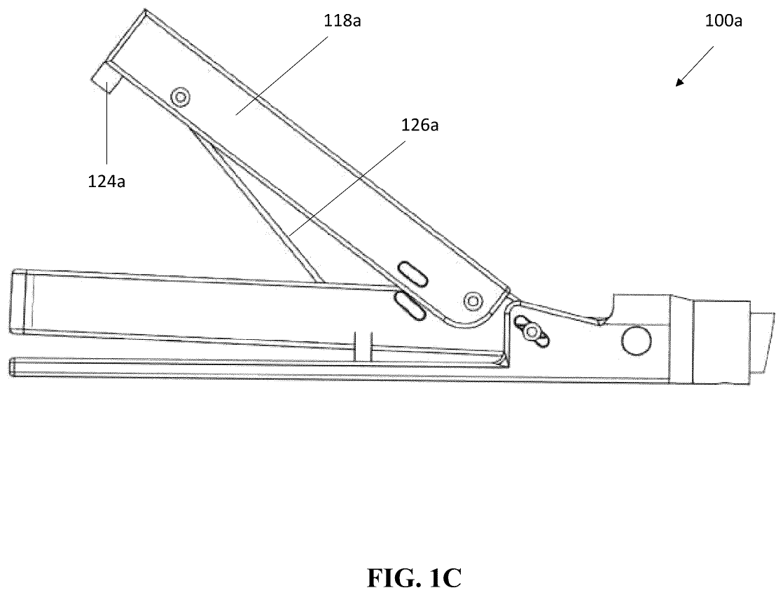

FIG. 1A-1C show an exemplary fastener dispenser 100 (shown as 100a) for dispensing a fastener 200 (shown as 200a) to fixate tissue and/or implant according to various implementations.

As shown in the example of FIG. 1A, the fastener dispenser 100 includes a first jaw 102 (shown as 102a), which includes a proximal end 104 (shown as 104a) and a distal end 106 (shown as 106a). A first jaw track 108 (shown as 108a) extends between the proximal end 104 and the distal end 106. The fastener dispenser 100 also includes a second jaw 110 (shown as 110a), which includes a proximal end 112 (shown as 112a) and a distal end 114 (shown as 114a). A second jaw track 116 (shown as 116a) extends between the proximal end 112 and the distal end 114. As provided herein, the fastener 200 further includes an elongated member 202 and a collar 210. In some implementations, the elongated member 202 is stored within the second jaw track 116, and the collar 210 is stored within the first jaw track 108.

In FIG. 1B and further shown in detail in FIG. 1C, the proximal end 104 of the first jaw 102 is pivotably coupled to the proximal end 112 of the second jaw 110. In use, the coupling allows the fastener dispenser 100 to move between an unclamped configuration, in which the first jaw 102 and the second jaw 110 have a relative spacing on opposite sides of the first layer and the second layer (e.g., opposite sides of the implant and patient's tissue), to a clamped configuration, in which the second jaw 110 is pivoted relative to the first jaw 102 to reduce the relative spacing between the first jaw 102 and the second jaw 110. In this configuration, the first jaw 102 and the second jaw 110 are forced together and exert a clamping force to hold the first layer relative to the second layer (e.g., hold the implant relative to the patient's tissue). In some implementations, the relative spacing in the unclamped configuration may be adjusted to accommodate variations in the second layer thickness (e.g., patient tissue thickness). In such implementations, a sliding adjustment mechanism may be integrated into the coupling of the first jaw 102 and second jaw 110 to adjust their relative spacing such that the spacing between the first jaw 102 and second jaw 110 in the clamped configuration is also adjusted.

Ejection Jaw. As shown in FIG. 1A, the fastener dispenser 100 further includes an ejection jaw 118 (shown as 118a) having a proximal end 120 (shown as 120a) and a distal end 122 (shown as 122a). The ejection jaw 118 is movably coupled to the second jaw 110. Specifically, the proximal end 120 of the ejection jaw 118 is pivotably coupled to the proximal end 112 of the second jaw 110. As shown in FIG. 1C, the ejection jaw 118 includes a protrusion 124 (shown as 124a) that extends from the distal end of the ejection jaw 118 toward the second track 116 of the second jaw 110. Specifically, the ejection jaw 118 shown is pivotably coupled to the second jaw 110 such that moving the ejection jaw 118 toward the second jaw 110 engages the protrusion 124 against the elongated member 202 stored in the second track 116 such that the protrusion 124 exerts a force upon the elongated member 202. Accordingly, moving the ejection jaw 118 toward the second jaw 110 causes the protrusion 124 to urge the elongated member 202 toward the collar 210 stored in the first jaw track 108 of the first jaw 102, thereby coupling the elongated member 202 with the collar 210 to form a coupled fastener 200. In other implementations, the protrusion 124 may extend from another aspect of the ejection jaw 118 other than the distal end 122, so long as the protrusion 124 engages the elongated member 202 and urges it toward the collar 210 as the ejection jaw 118 moves toward the second jaw 110.

While the ejection jaw 118 shown is movably coupled to the second jaw 110, this is not the only possible configuration. For example, in other configurations, the ejection jaw 118 may instead be movably coupled to the first jaw 102. In such implementations, the protrusion 124 may extend toward the first track 108 of the first jaw first jaw 102. Accordingly, the ejection jaw 118 may be instead moved toward the first jaw 102 to engage the protrusion 124 against the collar 210 stored in the first jaw track 108 such that the protrusion 124 exerts a force upon the collar 210, thereby urging the collar 210 toward the elongated member 202 stored in the second jaw track 116 of the second jaw 110 to form a coupled fastener 200.

Furthermore, while the ejection jaw 118 shown in FIG. 1C is pivotably coupled to the second jaw 110, the ejection jaw 118 may be movably coupled to the first jaw 102 or second jaw 110 in any configuration that allows the protrusion 124 to exert force on the elongated member 202 or collar 210, depending on which jaw the ejection jaw 118 is coupled to.

In some implementations, the protrusion 124 is not necessarily associated with an ejection jaw 118 or any other additional jaw, and may be any element that can be actuated to act upon at least one of the elongated member 202 or collar 210 so as to dispense the elongated member 202 or collar 210 from the second track 116 or first track 108, respectively, and move the elongated member 202 or collar 210 toward the other of the elongated member 202 or collar 210 to form a coupled fastener 200.

Biasing Member. In FIG. 1C, the ejection jaw 118 includes a biasing member 126 (shown as 126a) configured to urge the ejection jaw 118 toward the second jaw 110. In other implementations, the biasing member 126 may be configured to urge the ejection jaw 118 toward the first jaw 102. In various implementations, the biasing member 126 may further act upon one or more of the elongated member 202 or collar 210 stored within a respective first track 108 or second track 116.

In various implementations provided herein, the first jaw 102 may be configured for positioning posterior to the second layer (e.g., patient's target tissue), and the second jaw 110 may be configured for positioning anterior to the second layer (e.g., patient's target tissue). In implementations utilizing an ejection jaw 118, the ejection jaw 118 may thus also be configured for positioning anterior to both the second layer (e.g., the patient's target tissue) and the second jaw 110. In such implementations, the first jaw 102 may also be referred to as a “lower” jaw, the second jaw 110 may also be referred to as a “middle” jaw, and the ejection jaw 118 may also be referred to as an “upper” jaw, according to their relative orientations in use.

However, in various other implementations, the first jaw 102 may be configured for positioning anterior to the second layer (e.g., patient's target tissue), and the second jaw 110 may be configured for positioning posterior to the second layer (e.g., patient's target tissue). Furthermore, in implementations utilizing an ejection jaw 118, the ejection jaw 118 may be configured for positioning either anterior or posterior to the second layer (e.g., patient's target tissue) and thus anterior or posterior to one or both of the first jaw 102 and/or second 110 jaw. Accordingly, the first jaw 102, second jaw 110, and ejection jaw 118 may be positioned relative to one another in various combinations of orientations within the spirit of this disclosure. Thus, relative terms including “lower,” “middle,” and “upper,” as occasionally used herein denote arrangements characteristic of some, but not all, implementations.

In various implementations, one or more components of the fastener dispenser 100 may be formed from a polymeric material. In some implementations, one or more components of the fastener dispenser 100 may be formed from a metallic material, such as stainless steel.

### Example Fasteners

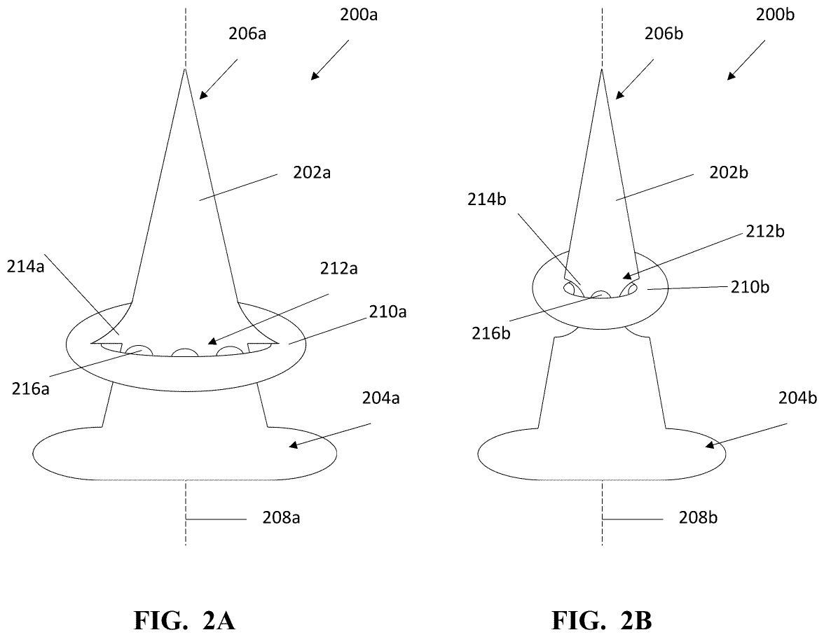

FIGS. 2A-2B show exemplary fasteners 200 (e.g., a “rivet” or a “tack”, shown as 200a and 200b). In the example shown in FIG. 2A, the fastener 200 includes an elongated member 202 (shown as 202a and 202b) and a collar 210 (shown as 210a and 210b). The elongated member 202 extends between a base end 204 (shown as 204a and 204b) and a penetrating end 206 (shown as 206a and 206b) and defines an elongated member central axis 208 (shown as 208a and 208b).

In FIG. 2A, the elongated member 202 includes a radially extending engagement feature 214 (shown as 214a). Specifically, FIG. 2A shows an elongated member 202 with a radially outwardly extending engagement feature 214a in the shape of a ridge. In some implementations, the radially outwardly extending engagement feature 214a may extend around all or only a part of the elongated member 202. Furthermore, the radially outwardly extending engagement feature 214a may include various other geometries configured to facilitate one-way coupling of the elongated member 202 with the collar 210. For example, other radially outwardly extending engagement feature 214a geometries include ribs, wings, bumps, or dimples extending radially outward from the elongated member 202.

In FIG. 2B, the radially extending engagement feature 214 (shown as 214b) of the elongated member 202 extends radially inward. Specifically, the exemplary implementation shown in FIG. 2B includes a circumferential groove defined in the elongated member 202. In some implementations, the radially inwardly extending engagement feature 214b may extend around all or only a part of the elongated member 202. In some implementations, the radially inwardly extending feature may be defined by various negative space geometries (e.g., impressions) configured to facilitate the one-way coupling of the elongated member 202 with the collar 210. Various implementations of the elongated member 202 may include a radially inwardly extending feature 214b with or without the addition of a radially outwardly extending feature 214a.

In both FIG. 2A and FIG. 2B, the collar 210 defines an opening 212 (shown as 212a and 212b) configured to receive the penetrating end 206 of the elongated member 202. In some implementations, the collar 210 further includes a radially extending engagement feature 216 (shown as 216a and 216b) configured to couple with the radially extending engagement feature 214 of the elongated member 202. Accordingly, the radially extending engagement feature 216 of the collar 210 may be radially inwardly extending or radially outwardly extending.

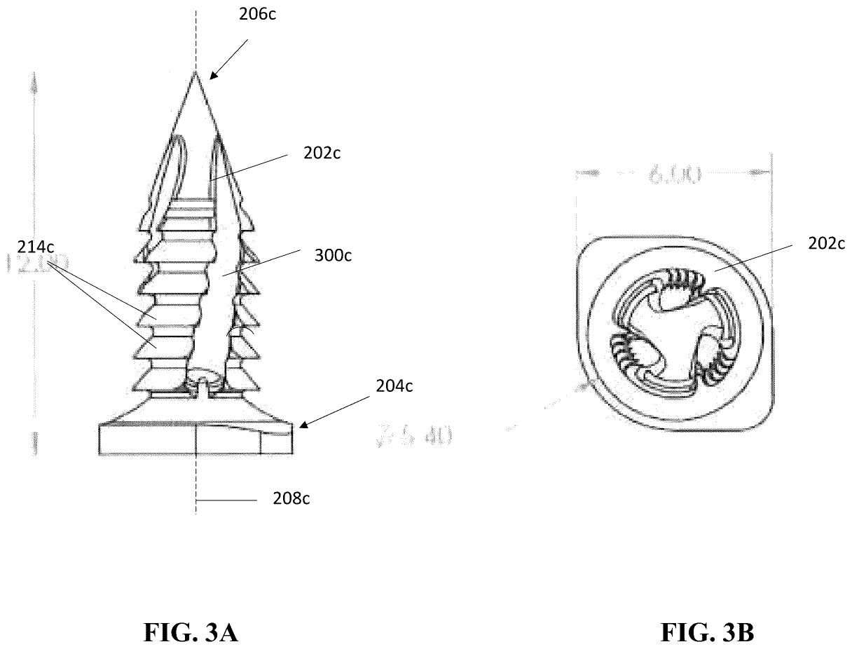

FIG. 3A and FIG. 3B show an exemplary elongated member 202 (shown as 202c), according to some implementations (side view and top view). As shown in FIG. 3A, the engagement feature 214 (shown as 214c) of the elongated member 202 may include a plurality of engagement features 214 spaced axially along the elongated member 202. Specifically, FIG. 3A shows a plurality of radially outwardly extending engagement features 214 formed as ridges. Each ridge is oriented parallel to the adjacent ridge. Furthermore, the elongated member 202 shown in FIG. 3A defines a set of three flutes 300 (shown as 300c) extending along the length of the elongated member 202. As shown, the flutes 300 are spaced equidistant from each other and rotate slightly about the central axis 208 (shown as 208c). Other implementations of the elongated member 202 may define a different number of flutes 300 or flutes 300 arranged with different relative spacing about the elongated member 202. Furthermore, in other implementations, the flutes 300 may be rotated more or less about the central axis 208.

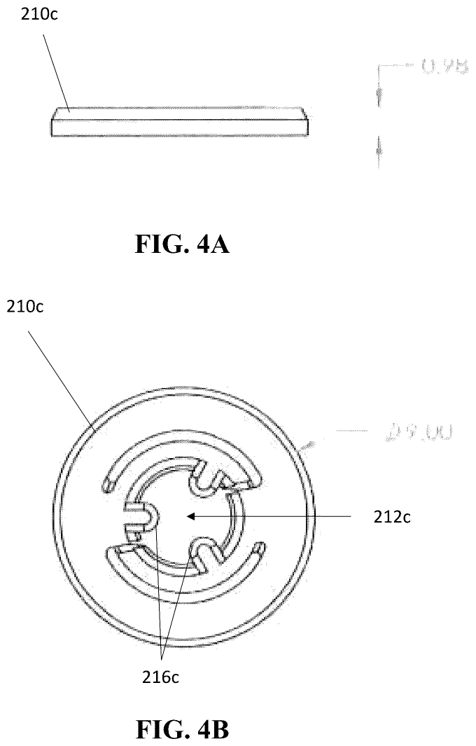

FIG. 4A and FIG. 4B show an exemplary collar 210 (shown as 210c), according to some implementations. As shown in FIG. 4B, the collar 210 defines a plurality of radially inwardly extending (i.e., protruding toward the opening 212c) engagement features 216 (shown as 216c) configured to engage between the radially outwardly extending engagement features 214 (e.g., ridge) of the elongated member 202 shown in FIG. 3A and FIG. 3B.

The fastener 200 provided herein may be configured to accommodate various second-layer thicknesses (e.g., patient tissue thicknesses). For example, the length of the elongated member 202 may be sized to span the combined thickness of the first layer and the second layer (e.g., the combined thickness of the patient's tissue and the implant). For example, the elongated member 202 shown in FIG. 3A has a total length of 12 mm. As provided herein and shown in FIG. 3A, the engagement feature 214 of the elongated member 202 may include a plurality of radially extending engagement features 214 spaced axially along the elongated member 202. In addition to facilitating coupling between the elongated member 202 and the collar 210, the plurality of radially extending engagement features axially spaced along the 202 allow the collar 210 to be coupled to the elongated member 202 at any one of several axial positions along the length of the elongated member 202, depending on the thickness of the intervening second layer (e.g., patient tissue).

In the exemplary implementations shown in FIGS. 2A-2B and FIG. 3A, the elongated member 202 is tapered toward the penetrating end 206. In some implementations, the taper forms an angle of 10 to 20 degrees from the central axis 208 of the elongated member 202. In other implementations, the taper forms an angle of 30 to 40 degrees from the central axis 208 of the elongated member 202. Furthermore, the penetrating end 206 is configured to narrow to a tip that is capable of piercing both the first layer and the second layer (e.g., piercing both the implant and the patient's tissue). In some exemplary implementations, the penetrating end 206 of the elongated member 202 may have a diameter of 15 microns. However, in other implementations, the elongated member 202 may not be tapered.

### Elongated Member in Cartridge With Pivot Operation

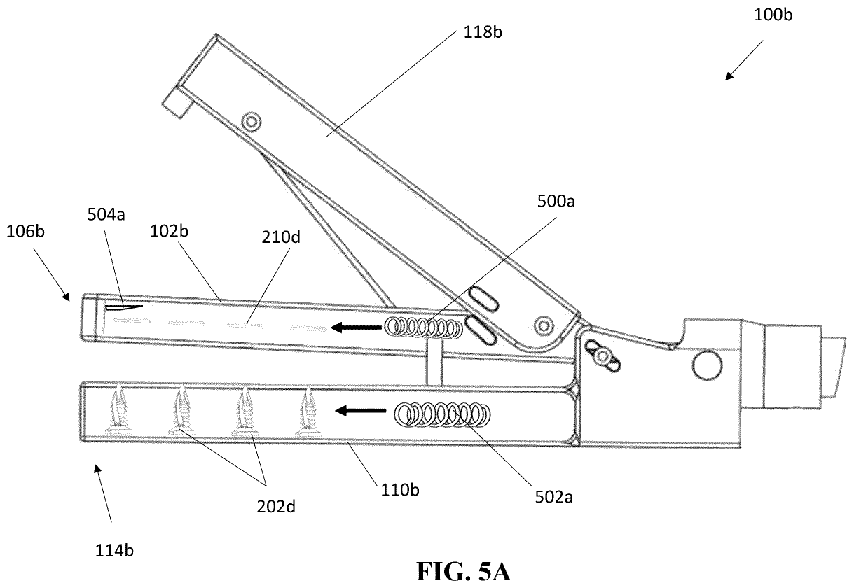

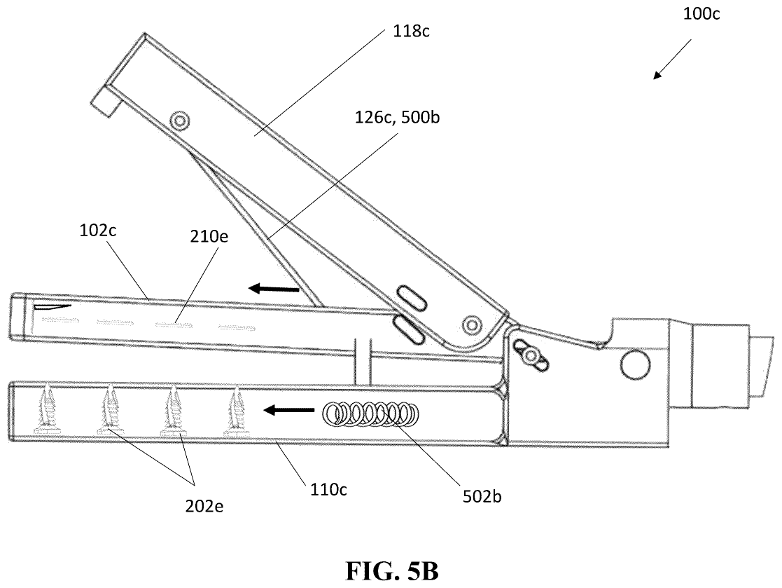

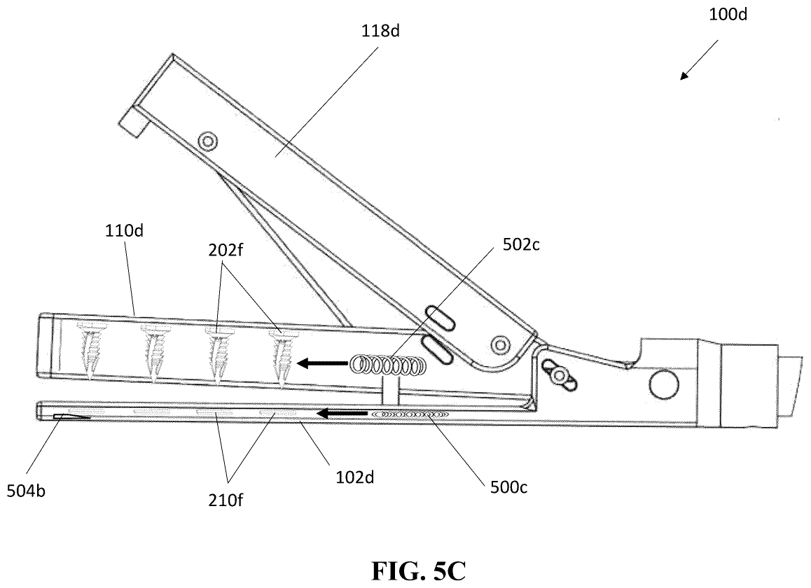

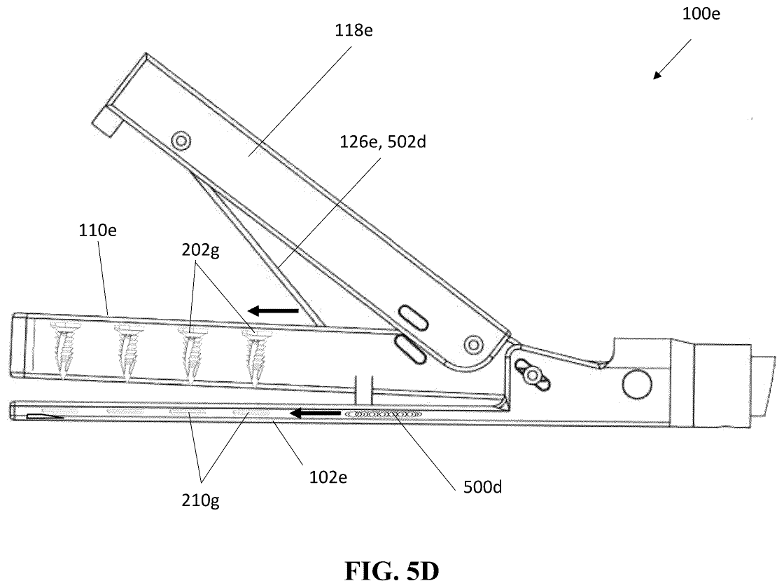

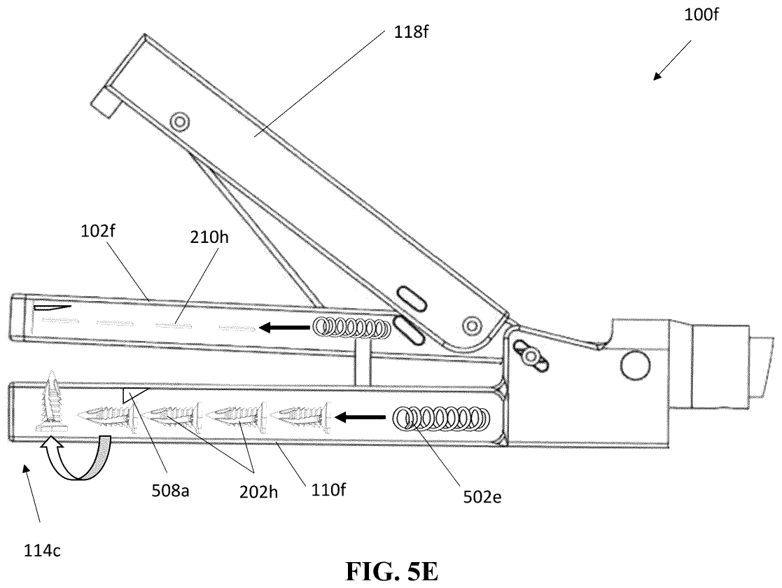

In some implementations, and as shown in FIG. 5A and 5B, the elongated member 202 is stored, in a cartridge, within the second jaw track 116 such that the elongated member 202 central axis 208 is parallel with or forms an acute angle with an extension axis of the second jaw track 116. In such implementations, the second jaw 110 further includes a pivoting feature configured to pivot the elongated member 202 when the elongated member 202 is dispensed such that the penetrating end 206 of the elongated member 202 extends toward the collar 210. Storing the elongated member 202 in this configuration allows for a reduction in the thickness of the second jaw 110, thereby overcoming the limited operating space available posterior to both the first layer and the second layer (e.g., posterior to both the implant and the target tissue). Thus, reducing the thickness of the second jaw 110 allows the second jaw 110 to be positioned posterior to the second layer (e.g., target tissue) as the “lower” jaw. In such implementations, the pivoting feature enables the elongated member 202 to be stored parallel or at an acute angle relative the extension axis of the second jaw track 116 and then pivot to be perpendicular with the second jaw track 116 in an orientation such that the penetrating end 206 extends anteriorly.

Orientations. As provided herein, the first jaw 102 and second jaw 110 may be configured for positioning in various orientations relative to the first layer and the second layer (e.g., relative to the implant and the patient's target tissue). In both FIG. 5A and FIG. 5B, the first jaw 102 and second jaw 110 (shown as 102b,c and 110b,c) are configured such that, in use, the first jaw 102 would be positioned anterior to both the first layer and the second layer (e.g., anterior to both the implant and the patient's target tissue), and the second jaw 110 would be positioned posterior to both the first layer and the second layer (e.g., posterior to both the implant and the patient's target tissue). Conversely, in both FIG. 5C and FIG. 5D, the first jaw 102 and second jaw 110 (shown as 102d,e and 110d,e) are configured such that, in use, the first jaw 102 would be positioned posterior to both the first layer and second layer (e.g., posterior to both the implant and the patient's target tissue), and the second jaw 110 would be positioned anterior to both the first layer and the second layer (e.g., anterior to both the implant and the patient's target tissue).

### Example Biasing Members

FIG. 5A shows fastener dispenser 100 (shown as 100b). As shown, the first jaw 102 (shown as 102b) includes a first biasing member 500 (shown as 500a) that urges the collar 210 (shown as 210d) toward the distal end 106 (shown as 106b) of the first jaw 102. As shown, the collar 210 includes a plurality of collars 210 (shown as 210d). In such implementations, the first biasing member 500 progressively advances the plurality of collars 210 toward the distal end 106 of the first jaw 102 as successive collars 210 are dispensed from the fastener dispenser 100. In some implementations, the biasing member 126 may also act as the first biasing member 500. For example, FIG. 5B shows fastener dispenser 100 (shown as 100c) with the biasing member 126 (shown as 126c, 500b) exerting a force on the plurality of collars 210 (shown as 210e), urging the plurality of collars 210 toward the distal end 106 of the first jaw 102. Of course, the relative orientation of the first jaw 102 (and accompanying first biasing member 500) and second jaw 110 (and accompanying second biasing member 502) be reversed relative to FIG. 5A, as shown, for example, in the fastener dispenser 100 (shown as 100d) in FIG. 5C.

As shown in FIGS. 5A and 5C, the second jaw 110 (shown as 110b) includes a second biasing member 502 (shown as 502a) that urges the elongated member 202 toward the distal end 114 of the second jaw 110. As shown, the elongated member 202 (shown as 202d) includes a plurality of elongated members 202. In such implementations, the second biasing member 502 progressively advances the plurality elongated members 202 toward the distal end 114 (shown as 114b) of the second jaw 110 as successive elongated members 202 are dispensed from the fastener dispenser 100. In some implementations, the biasing member 126 may also act as the second biasing member 502.

FIG. 5D shows fastener dispenser 100 (shown as 100e) where the relative orientation of the first jaw 102 (and accompanying first biasing member 500) and second jaw 110 (and accompanying second biasing member 502) are reversed relative to FIG. 5A. For example, FIG. 5D shows the biasing member 126 (shown as 126e, 502d) exerting a force on the plurality of elongated members 202, urging the plurality of elongated members 202 toward the distal end 114 of the second jaw 110.

### Example Cutting Blade

In various implementations, the fastener 200 may further include a cutting blade 504. As shown in FIG. 5A, the cutting blade 504 (shown as 504a) is coupled to the first jaw 102. As provided herein, the cutting blade is configured to remove a portion of the penetrating end 206 of the elongated member 202 on a side of the collar 210 opposite the base end 204 of the elongated member 202 after the elongated member 202 is coupled to the collar 210. Specifically, the cutting blade shown in FIG. 5A is configured to cut the penetrating end 206 after the penetrating end 206 passes anteriorly through the first layer and the second layer (e.g. anteriorly through the implant and the patient's tissue) and is coupled with the collar 210. As shown for example in FIG. 5C, the cutting blade 504 (shown as 504b) may be oriented differently relative to the orientation of cutting blade 504a, depending on the orientation of the first jaw 102. In other implementations, the cutting blade may be coupled to the first jaw 102 such that it can cut the penetrating end 206 after the penetrating end 206 passes posteriorly through the first layer and the second layer (e.g. posteriorly through the implant and the patient's tissue) and is coupled with the collar 210. Although the cutting blade shown in FIG. 5A is disposed within the first jaw 102, in some implementations, the cutting blade may be disposed adjacent to the first jaw 102, such as between the first jaw 102 and the second jaw 110.

### Example Reduced Sized Device

In some implementations, the elongated member 202 is stored within the second jaw track 116 such that the elongated member 202 central axis 208 is parallel with or forms an acute angle with an extension axis of the second jaw track 116. For example, FIG. 5E shows the elongated member 202 (shown as 202h) being stored such that the central axis 208 is parallel with the extension axis of the second jaw track 116 of the second jaw 110 (shown as 110f). In such implementations, the second jaw 110 further includes a pivoting feature 506 (shown as 506a) configured to pivot the elongated member 202 when the elongated member 202 is dispensed such that the penetrating end 206 of the elongated member 202 extends toward the collar 210. As shown in FIG. 5E, for example, advancing the elongated member 202 along the second jaw track 116 toward the distal end 114 (shown as 114c) causes the elongated member 202 to contact the pivoting feature 506. In some examples, including the illustrated implementation, the pivoting feature 506 contacts and catches a portion of the base end 204 of an elongated member, thereby pivoting the elongated member 202 90° from an orientation parallel with the extension axis of the second jaw track 216 to an orientation perpendicular to the extension axis of the second jaw track 216. As shown, pivoting the elongated member 202 aligns the elongated member 202 with the opening 212 of collar 210 (shown as 210h). The pivoting feature 506 shown in FIG. 5E includes an angled wedge-shaped protrusion extending from an inner aspect of the second jaw 110. However, in other implementations, the pivoting features 506 may include other geometries, such as bumps or shoulders that extend toward the elongated member 202. In still yet other implementations, the pivoting feature 506 may include a hinge configured to rotate the elongated member 202 as described herein.

Storing the elongated member 202 in this fashion allows for a reduction in the thickness of the second jaw 110, thereby overcoming the limited operating space available posterior to both the first layer and the second layer (e.g., posterior to both the implant and the target tissue). Thus, reducing the thickness of the second jaw 110 allows the second jaw 110 to be positioned posterior to the second layer (e.g., target tissue) as the “lower” jaw. In such implementations, the pivoting feature enables the elongated member 202 to be stored parallel or at an acute angle relative the extension axis of the second jaw track 116 and then pivot to be perpendicular with the second jaw track 116 in an orientation such that the penetrating end 206 extends anteriorly.

### Example Cartridges for Collar

In various implementations, the surgical fastening system further includes a collar cartridge configured to store and dispense the collar 210. In some implementations the collar cartridge is configured to store a plurality of collars 210. In some implementations, the first jaw track 108 is configured to receive the collar cartridge. In some implementations, the first biasing member is configured to urge the collar cartridge toward the distal end 106 of the first jaw 102. In some implementations, the collar cartridge includes an opening through which the protrusion 124 may engage the collar 210 and urge the collar 210 out of the collar cartridge and toward the elongated member 202.

In various implementations, the surgical fastening system further includes an elongated member cartridge configured to store and dispense the elongated member 202. In some implementations the elongated member cartridge is configured to store a plurality of elongated members 202. In some implementations, the second jaw track 116 is configured to receive the elongated member cartridge. In some implementations, the second biasing member is configured to urge the elongated member cartridge toward the distal end 114 of the second jaw 110. In some implementations, the elongated member cartridge includes an opening through which the protrusion 124 may engage the elongated member 202 and urge the elongated member 202 out of the elongated member cartridge and toward the collar 210.

### Dispensing Blade (Elongated Member as an Extension of Fastener Dispenser)

In various implementations, the elongated member 202 may be formed integrally with (i.e., from the same bulk of material) the second jaw 110 and extend toward the first jaw 102. Such implementations may include a plurality of elongated members 202 formed integrally with the second jaw 110. In such implementations, the elongated member 202 dispensing the elongated member 202 from the second jaw 110 involves physically severing the elongated member 202 from the first jaw 102. Accordingly, the fastener dispenser 100 may further include a dispensing blade configured to remove the elongated member 202 from the second jaw 110.

### Example Housing

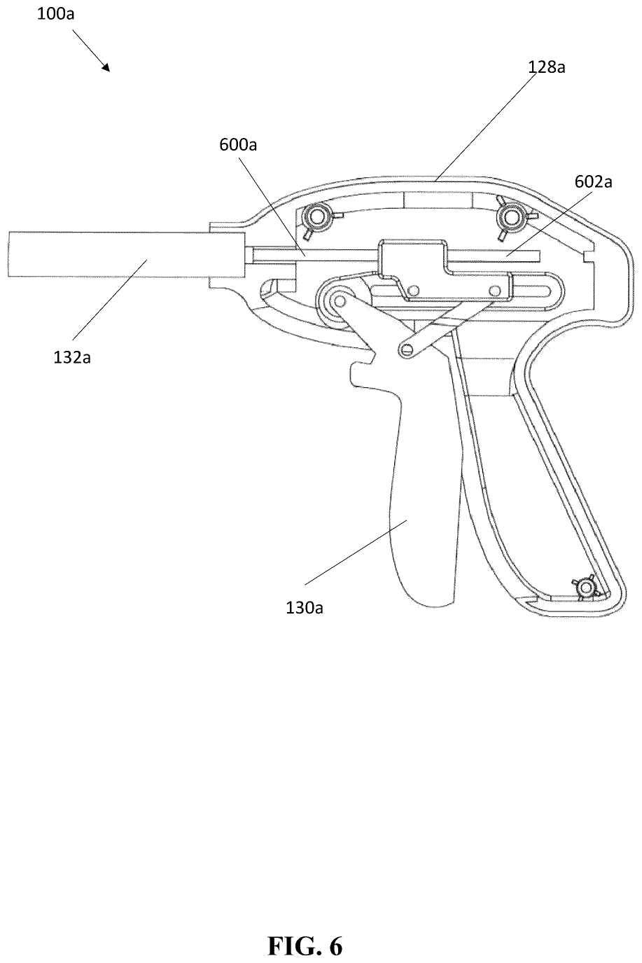

Referring generally to the figures, FIG. 1A and FIG. 1B show a fastener dispenser 100 including a housing 128 (shown as 128a). FIG. 6 shows a detailed partial cross-sectional view of the housing 128 shown in FIG. 1B. As shown in FIG. 6, the housing 128 includes a first actuator 600 (shown as 600a) movable between a first unengaged position and a first engaged position. Movement of the first actuator 600 between the first unengaged position and the first engaged position changes the relative spacing between the first jaw 102 and the second jaw 110 such that the fastener dispenser 100 moves between the unclamped configuration and the clamped configuration, respectively. In some implementations, moving the first actuator 600 from the first unengaged position to the first engaged position reduces the relative spacing between the first jaw 102 and the second jaw 110. As provided herein, the first actuator 600 urges the first jaw 102 and second jaw 110 together so that the jaws may exert a clamping force to hold the first layer and the second layer (e.g., hold the implant relative to the patient's tissue). Conversely, in some implementations, moving the first actuator 600 from the first engaged position to the first unengaged position increases the relative spacing between the first jaw 102 and the second jaw 110, thereby releasing the first layer and/or second layer from the first jaw 102 and second jaw 110.

In various implementations, the housing 128 further includes a second actuator 602 (shown as 602a) movable between a second unengaged position and a second engaged position. Movement of the second actuator 602 between the second unengaged position and the second engaged position changes the relative spacing between the ejection jaw 118 and one of the first jaw 102 or the second jaw 110. As provided herein, the second actuator 602 urges the ejection jaw 118 toward one of the elongated member 202 or the collar 210 stored within the second jaw 110 or first jaw 102, respectively. Specifically, moving the second actuator 602 between the second unengaged position and the second engaged position engages the protrusion 124 against one of the elongated member 202 or the collar 210 such that the protrusion 124 exerts a force upon the elongated member 202 or collar 210. Accordingly, moving the second actuator 602 between the second unengaged position and the second engaged position urges one of the elongated member 202 or the collar 210 toward the other of the elongated member 202 or the collar 210 to couple the elongated member 202 and the collar 210, thereby forming a coupled fastener 200. Furthermore, in various implementations, the force exerted by the protrusion 124 on the elongated member 202 or the collar 210 also dispenses the elongated member 202 or the collar 210 from the second jaw 110 or first jaw 102, respectively.

### Example Handle

As shown in the example of FIG. 6, the housing 128 further includes a handle 130 (shown as 130a) configured to move the first actuator 600 between the first unengaged position and the first engaged position.

In some implementations, the handle 130 is further configured to move the second actuator 602 between the second unengaged position and the second engaged position. As such, in some implementations, the first actuator 600 and the second actuator 602 are sequentially moved by a single handle 130 as the handle 130 is engaged. For example, in some implementations, engagement of the handle 130 between a first position and a second position moves the first actuator 600 between the first unengaged position and the first engaged position and engagement of the handle 130 between a second position and a third position moves the second actuator 602 between the second unengaged position and the second engaged position. In some implementations, a second handle or other input feature may be engaged to actuate one of the first actuator 600 or second actuator 602.

As shown, for example, in FIG. 1A, the surgical fastening system further includes a hollow shaft 132 (shown as 132a) extending from the housing 128 to the fastener dispenser 100. The hollow shaft 132 is sized and configured to allow components of the first actuator 600 and second actuator 602 to move therewithin. As shown, the hollow shaft 132 is fixed to the first jaw 102. Specifically, the first jaw 102 is both rotationally and pivotably fixed to the hollow shaft 132. Accordingly, tilting or maneuvering the housing 128 causes corresponding tilting or maneuvering of the first jaw 102. Furthermore, the position of the first jaw 102 relative to the hollow shaft 132 is maintained when the first actuator 600 is moved between the first unengaged position and the first engaged position. Instead, it is the second jaw 110 that moves relative to the first jaw 102 and the hollow shaft 132 as the fastener dispenser 100 moves between unclamped and clamped configurations.

However, in other implementations, the hollow shaft 132 may be fixed to the second jaw 110. In such implementations, the second jaw 110 is both rotationally and pivotably fixed to the hollow shaft 132. Accordingly, tilting or maneuvering the housing 128 causes corresponding tilting or maneuvering of the second jaw 110. Furthermore, the position of the second jaw 110 relative to the hollow shaft 132 is maintained when the first actuator 600 is moved between the first unengaged position and the first engaged position. Instead, it is the first jaw 102 that moves relative to the second jaw 110 and the hollow shaft 132 as the fastener dispenser 100 moves between unclamped and clamped configurations.

### Connect/Disconnect Operation

In various implementations, the fastener dispenser 100 is detachably coupled to the hollow shaft 132 for disposal or replacement. In some implementations, the hollow shaft 132 is specifically detachably coupled to the first jaw 102. In other implementations, the hollow shaft 132 is specifically detachably coupled to the second jaw 110.

### Example Clinical Applications

The surgical fastening system provided herein may be used in various minimally invasive and open surgical procedures. In various use cases provided herein, the surgical fastening system is employed to secure a surgical mesh corresponding to a first layer) to a target tissue (corresponding to a second layer) in the course of surgical hernia repair, such as the repair of ventral and incisional hernias. In such uses, the surgical mesh is a hernia mesh, and the target tissue corresponds to a hernia defect site. However, the surgical fastening system may be utilized to secure other soft implants to other tissues or certain layers of tissue to other layers of tissue in the course of other surgical procedures. In some implementations, the implant is formed from an absorbable material, such as synthetic polyester copolymer derived from lactic and glycolic acid. In other implementations, the implant may be nonabsorbable.

In various other use cases, the surgical fastening system may be employed to fix one segment of patient tissue (corresponding to a first layer) to another segment of patient tissue (corresponding to a second layer).

And yet other use cases, the surgical fastening system may be employed to fix one implant (corresponding to a first layer) to another implant (corresponding to a second layer) or one segment of an implant (corresponding to a first layer) to another segment of the same implant (corresponding to a second layer).

In use, the elongated member 202 is configured to engage with the opening 212 of collar 210 to form a coupling therebetween, thereby forming a two-component coupled fastener 200. As provided herein, the fastener 200 may be implemented in a surgical fastening system that includes a fastener dispenser 100 according to any of the implementations provided herein, wherein the fastener 200 is stored in and dispensed from the fastener dispenser 100. Thus, in some implementations, the elongated member 202 and collar 210 of the fastener 200 are coupled by the fastener dispenser 100.

### Example Methods of Use

In another aspect, this disclosure relates to a method of fixing a first layer (e.g., an implant) to a second layer (e.g., a patient's tissue). In various implementations, the method includes: (i) positioning a first jaw 102 or a second jaw 110 of a fastener dispenser 100 posterior to a patient's tissue and an implant; (ii) positioning the other of the first jaw 102 or second jaw 110 anterior to the first and second layers; (iii) reducing a relative spacing between the first jaw 102 and the second jaw 110 so as to clamp the first layer and second layer together and hold the first layer and second layer relative to each other; (iv) penetrating the first layer and second layer with an elongated member 202 stored in the second jaw 110; and (v) coupling the elongated member 202 with a collar 210 stored in the first jaw 102 to form a coupled fastener 200 spanning the thickness of the first layer and the second layer, thereby fixing the first layer relative to the second layer. For example, the method may entail fixing the implant relative to the patient's tissue.

The method further includes dispensing the elongated member 202 and collar 210 from the second jaw 110 and first jaw 102, respectively. In some implementations, the steps of dispensing the elongated member 202 and/or collar 210 may occur after the coupled fastener 200 is formed. In such cases, the elongated member 202 and collar 210 are concurrently dispensed from the second jaw 110 and the first jaw 102 as a coupled fastener 200. In other implementations, the elongated member 202 and/or collar 210 may be dispensed from the second jaw 110 and first jaw 102, respectively, prior to coupling.

In various implementations, the method of dispensing at least one of the elongated member 202 and collar 210 from the second jaw 110 and first jaw 102, respectively, includes engaging a protrusion 124 against the elongated member 202 or the collar 210, thereby urging the elongated member 202 or the collar 210 out of the second jaw track 116 or first jaw track 108 in which it is stored In various implementations, the protrusion 124 extends from an ejection jaw 118 that is movably coupled to one of the first jaw 102 or second jaw 110. In such implementations, the protrusion 124 extends toward the track of the first jaw 102 or second jaw 110, to which the ejection jaw 118 is coupled.

In various implementations, the method includes: (i) providing a fastener dispenser 100 including: a first jaw 102 having a proximal end 104 and a distal end 106, the first jaw 102 defining a first jaw track 108 therein extending between the proximal end 104 and the distal end 106; a second jaw 110 having a proximal end 112 and a distal end 114, the second jaw 110 defining a second jaw track 116 therein extending between the proximal end 112 and the distal end 114; (ii) providing a fastener 200 including: an elongated member 202 and a collar 210, the elongated member spanning the combined thickness of the first layer and the second layer (e.g., the combined thickness of the patient's tissue and the implant), wherein the elongated member 202 extends between a base end 204 and a penetrating end 206 and defines an elongated member central axis 208; wherein the collar 210 defines an opening 212 configured to receive the penetrating end 206 of the elongated member 202; and wherein the elongated member 202 is stored within the second jaw track 216 and the collar 210 is stored within the first jaw track 216; (iii) positioning the fastener dispenser 100 so that the first layer and second layer (e.g., the implant and the patient's tissue) are disposed between the first jaw 102 and the second jaw 110; (iv) moving the fastener dispenser 100 from an unclamped configuration, in which the first jaw 102 and the second jaw 110 have a relative spacing on opposite sides of the first and second layer (e.g., opposite sides of the implant and patient's tissue), to a clamped configuration, in which the second jaw 110 is pivoted relative to the first jaw 102 to reduce the relative spacing between the first jaw 102 and the second jaw 110, thereby holding the first layer relative to the second layer (e.g., the implant relative to the patient's tissue); and (v) coupling the elongated member 202 with the collar 210, thereby clamping the first layer and the second layer (e.g., clamping the implant and the patient's tissue) between the base end of the elongated member 202 and the collar 210, thereby fixing the first layer relative to the second layer (e.g., fixing an implant relative to the patient's tissue).

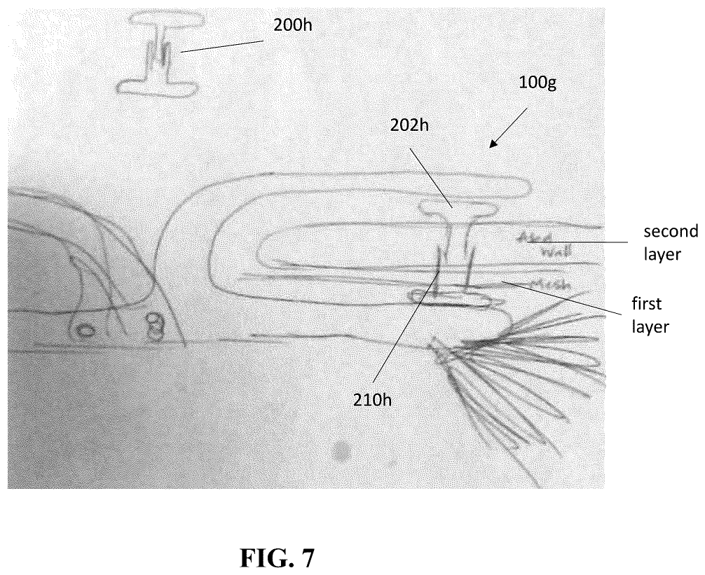

Accordingly, moving the fastener dispenser 100 between the unclamped and clamped configurations involves moving the first jaw 102 and second jaw 110 relative to one another. Specifically, moving the fastener dispenser 100 from the clamped configuration to the unclamped configuration involves reducing a relative spacing between the first jaw 102 and the second jaw 110. FIG. 7 shows an exemplary fastener dispenser 100 (shown as 100f) in the clamped configuration. As shown, in the clamped configuration, the second jaw 110 is pivoted relative to the first jaw 102 such that the first jaw 102 and second jaw 110 exert clamping forces toward one another to hold the patient's tissue and implant therebetween. In the unclamped configuration, the first jaw 102 and the second jaw 110 do not exert forces toward one another, thereby allowing relative movement of the fastener dispenser 100, the patient's tissue, and the implant (i.e., before fastening) or allowing the release of the fixed implant and patient's tissue (i.e., after forming the coupled fastener 200).

As shown, for example, in FIG. 6, a first actuator 600 (shown as 600a) is provided in the housing 128 to move the fastener dispenser 100 between the clamped configuration and the unclamped configuration. As provided herein, moving the first actuator 600 from a first unengaged position to a first engaged position reduces the relative spacing between the first jaw 102 and the second jaw 110, thereby moving the fastener dispenser 100 from the unclamped configuration to the clamped configuration. As provided herein, the first actuator 600 urges the first jaw 102 and second jaw 110 together so that the first jaw 102 and second jaw 110 exert clamping forces to hold the implant relative to the patient's tissue. Thus, the first actuator 600 is configured to generate sufficient force to maintain the fastener dispenser 100 in the clamped configuration.

The method further includes dispensing the elongated member 202 and collar 210 from the second jaw 110 and first jaw 102, respectively. In some implementations, the steps of dispensing the elongated member 202 and/or collar 210 may occur after the coupled fastener 200 is formed. In such cases, the elongated member 202 and collar 210 are concurrently dispensed from the second jaw 110 and the first jaw 102 as a coupled fastener 200. In other implementations, the elongated member 202 and/or collar 210 may be dispensed from the second jaw 110 and first jaw 102, respectively, prior to coupling.

In various implementations, the method of dispensing at least one of the elongated member 202 and collar 210 from the second jaw 110 and first jaw 102, respectively, includes engaging a protrusion 124 against the elongated member 202 or the collar 210, thereby urging the elongated member 202 or the collar 210 out of the second jaw track 116 or first jaw track 108 in which it is stored. In various implementations, the protrusion 124 extends from an ejection jaw 118 that is movably coupled to one of the first jaw 102 or second jaw 110. In such implementations, the protrusion 124 extends toward the track of the first jaw 102 or second jaw 110, to which the ejection jaw 118 is coupled.

In various implementations, urging the elongated member 202 and collar 210 toward each other includes penetrating the elongated member 202 through the relevant layers and engaging the collar 210 with the elongated member 202 such that the elongated member 202 is received within an opening 212 of the collar 210. As provided above, the collar 210 may be indexed at several locations along the length of the elongated member 202 to generate optimal pressure on the layers.

In various implementations, coupling the elongated member 202 with the collar 210 further includes moving the ejection jaw 118 toward the first jaw 102 or second jaw 110 to which it is coupled to engage the protrusion 124 against the elongated member 202 or collar 210 stored in the track of the first jaw 102 or second jaw 110. Thus, the protrusion 124 urges the elongated member 202 or collar 210 toward the other of the elongated member 202 or collar 210 to couple the elongated member 202 and collar 210. In some implementations, the protrusion 124 is not necessarily associated with an ejection jaw 118 or any other additional jaw. In such implementations, the protrusion 124 may be any element that can be actuated to act upon at least one of the elongated member 202 or collar 210 so as to dispense the elongated member 202 or collar 210 from the second track 116 or first track 108, respectively, and move the elongated member 202 or collar 210 toward the other of the elongated member 202 or collar 210 to form a coupled fastener 200.

In various implementations, as shown, for example, in FIG. 6, a second actuator 602 is provided in the housing 128 to move the fastener dispenser 100 between a second unengaged position and a second engaged position to engage the protrusion 124 against the he elongated member 202 or the collar 210, thereby urging the elongated member 202 or the collar 210 toward the other of the elongated member 202 or collar 210 to couple the elongated member 202 and the collar 210, thereby forming a coupled fastener 200. Specifically, moving the second actuator 602 between the second unengaged position and the second engaged position engages the protrusion 124 against one of the elongated member 202 or the collar 210 such that the protrusion 124 exerts a force upon the elongated member 202 or collar 210. Thus, the second actuator 602 is configured to generate sufficient force to couple the elongated member 202 and the collar 210 to form a coupled fastener 200. In implementations wherein the protrusion 124 is a component of an ejection jaw, moving the second actuator 602 between the second unengaged position and the second engaged position changes the relative spacing between the ejection jaw 118 and at least one of the first jaw 102 or second jaw 110.

Furthermore, in various implementations, the force exerted by the protrusion 124 on the elongated member 202 or the collar 210 also dispenses the elongated member 202 or the collar 210 from the second jaw 110 or first jaw 102, respectively.

In some implementations, coupling the elongated member 202 with the collar 210 further includes urging the penetrating end 206 of the elongated member 202 through the implant and the patient's tissue toward the collar 210 and engaging the elongated member 202 with the opening 212 of the collar 210. Thus, the second actuator 602 is configured to generate sufficient force to penetrate the patient's relevant tissues and the implant with the penetrating end 206 of the elongated member 202.

As provided herein, the elongated member 202 may be stored within the second jaw track 116 in a storage orientation, such that the elongated member central axis 208 is parallel with or forms an acute angle with an extension axis of the second jaw track 116. In such implementations, the method further includes pivoting the elongated member from the storage orientation to a dispensing orientation, in which the elongated member central axis 208 is aligned with opening 212 of the collar 210.

The surgical fastening system provided herein may be used in various minimally invasive and open surgical procedures. In various use cases provided herein, the surgical fastening system is employed to fix a surgical mesh to a target tissue in the course of surgical hernia repair, such as the repair of ventral and incisional hernias. In such uses, the surgical mesh is a hernia mesh, and the target tissue corresponds to a hernia defect site. However, the surgical fastening system may be utilized to fix other soft implants to other tissues in the course of other surgical procedures. In some implementations, the implant is formed from an absorbable material, such as synthetic polyester copolymer derived from lactic and glycolic acid. In other implementations, the implant may be nonabsorbable.

In various implementations provided herein, the first jaw 102 may be positioned posterior to the patient's target tissue, and the second jaw 110 may be positioned anterior to the patient's target tissue. In implementations utilizing an ejection jaw 118, the ejection jaw 118 may thus also positioned anterior to both the patient's target tissue and the second jaw 110.

However, in various other implementations, the first jaw 102 is positioned anterior to the patient's target tissue, and the second jaw 110 is positioned posterior to the patient's target tissue. Furthermore, in implementations utilizing an ejection jaw 118, the ejection jaw 118 may be positioned either anterior or posterior to the patient's target tissue and thus anterior or posterior to one or both of the first jaw 102 and/or second 110 jaw. Accordingly, the first jaw 102, second jaw 110, and ejection jaw 118 may be positioned relative to one another in various combinations of orientations within the spirit of this disclosure.

Accordingly, provided herein is a surgical fastening system including a fastener dispenser 100 for storing, dispensing, and coupling a two-part fastener 200. The system enables surgeons to precisely and efficiently fix an implant to a patient's tissue, thereby reducing surgical time and improving implant fixation outcomes.

### Experimental Results and Additional Examples

A study was conducted to develop systems and methods for fixing soft prostheses or tissues to fascial tissues in patients with ventral hernias through open or minimally invasive methods. In some aspects, a surgical fastener system for the fixation of soft tissues or soft prostheses during surgical procedures, such as ventral hernia repairs, is provided.

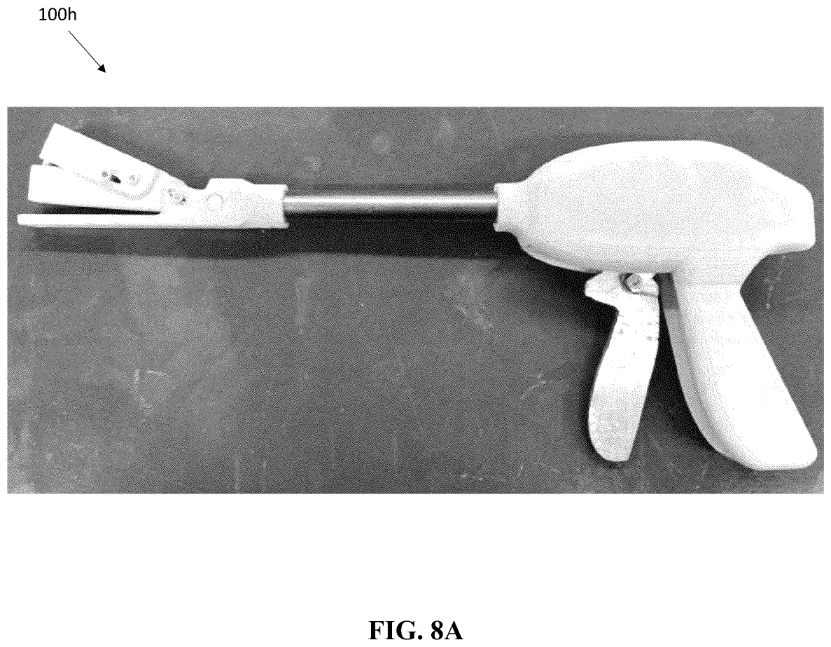

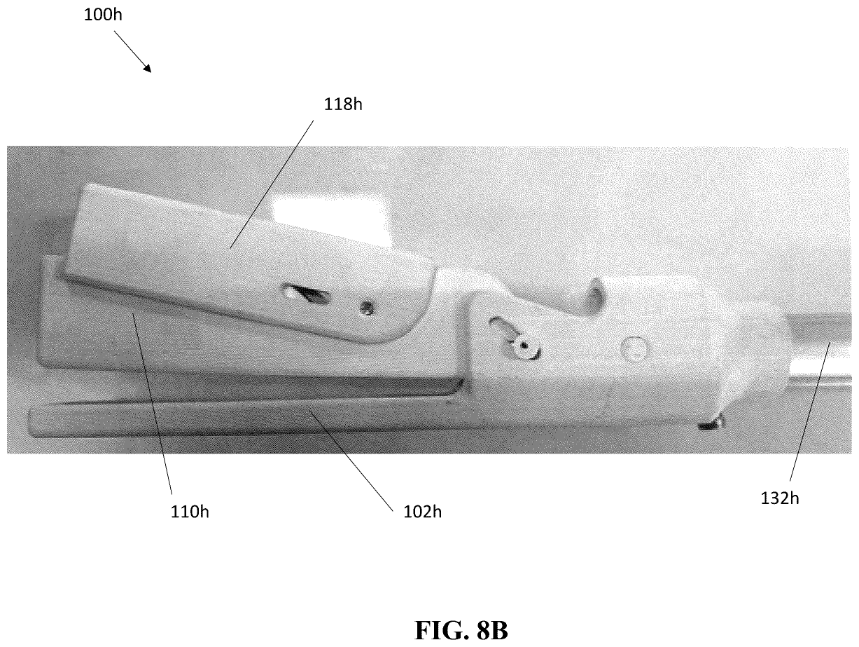

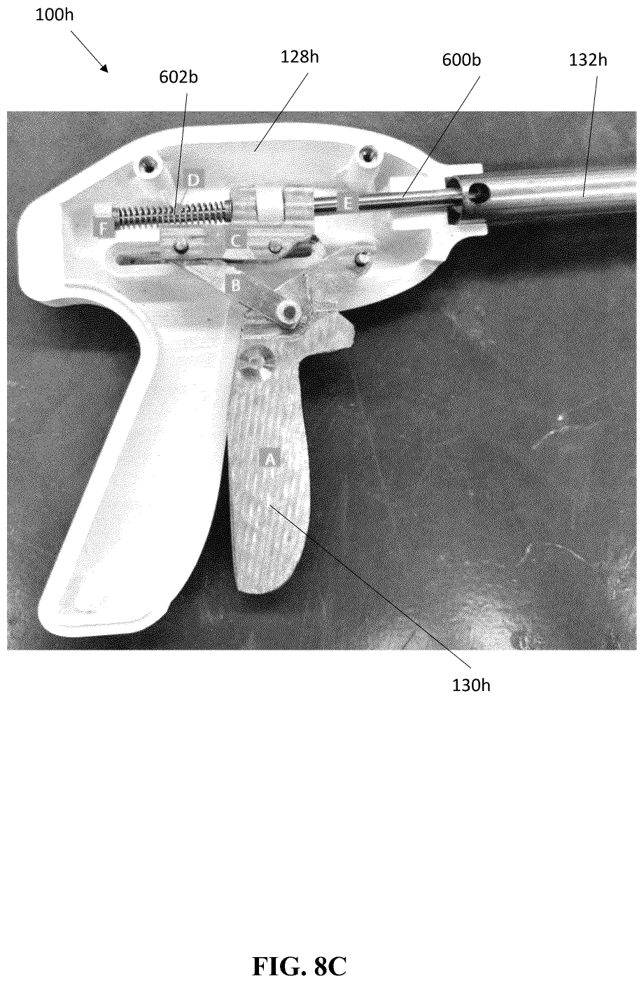

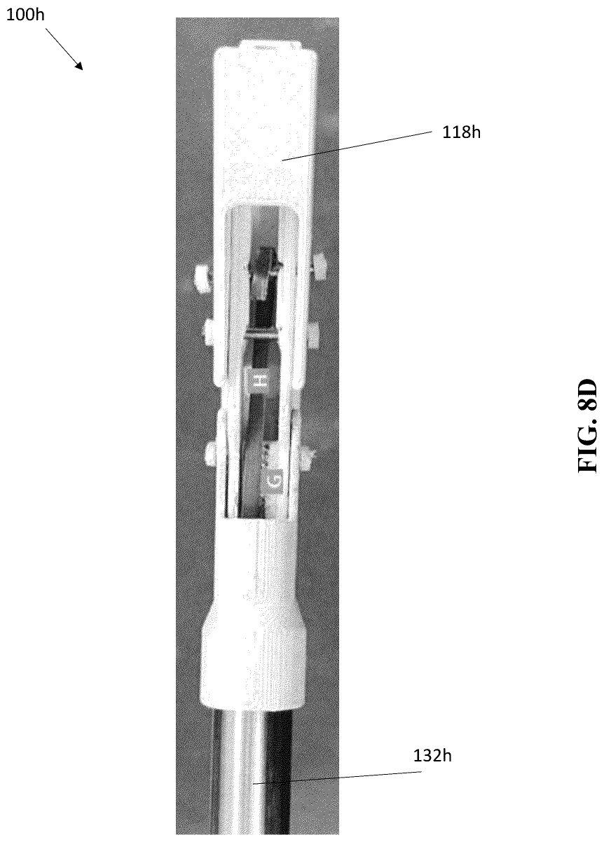

Example Fastener Dispenser. FIG. 8A-8D show various views of an exemplary fastener dispenser 100 (shown as 100g) configured to hold and dispense a two-part fastener in accordance with an illustrative embodiment. In this implementation, tissue clamping between the first jaw 102 and the second jaw 110, as well as fastener 200 dispensing mediated by engagement of the protrusion 124 of the ejection jaw 118 are each actuated by a single handle 130, wherein tissue clamping is initiated by actuation of the handle 130 and dispensing of the fastener 200 is initiated once the handle 130 passes a pre-determined transition point in its range of motion.

As shown in FIG. 8B, a slot and pin mechanism couples the first jaw 102 and the second jaw 110, and a separate slot and pin mechanism couples the second jaw 110 and ejection jaw 118. In implementations where the first jaw 102 and second jaw 110 are oriented opposite relative to their positions in FIG. 8B, the ejection jaw 118 may instead be coupled to the first jaw 102. With two separate pin and slot mechanisms, independent movement of the jaws is achieved. By pulling each through pin toward the housing, the pin slides along the overlapping slots and closes the corresponding jaw. This independent jaw motion allows for two phases of fastener 200 delivery: layer clamping and fastener 200 dispense.

As shown in FIG. 8C, the single handle 130 (A) controls the movement of a linkage (B) and carriage (C) system responsible for moving the second actuator (e.g., “push rod”) 602 (D) and first actuator (e.g., “push tube” 600) (E) components. During the first phase of handle 130 motion (handle shown as 130h), the push rod 602 and push tube 600 components move as a unit until the stopper (F) contacts the back wall of the housing shell 128; this action marks the transition from clamping to fastener dispense phases. As the handle is continually pressed, the stopper remains in contact with the back wall-ensuring clamp force is maintained-and the spring is compressed, allowing the push rod 602 to move relative to the push tube and initiate fastener 200 dispense.

The push rod 602 and push tube 600 interface with other linkages in the head of the deployment tool, shown in FIG. 8D. To control the tissue clamping motion, the push rod 602 connects to a solid linkage (G) and through-pin. Tack dispense is controlled by the push tube 600, which attaches to a metal ribbon (H) and corresponding through-pin. In some implementations, lubricants may be used to reduce friction between the various components.

As shown for example in FIG. 8C, the fastener dispenser 100 includes a two-stage mechanical delivery system disposed within a housing 128 (shown as 128h) and an ergonomic handle 130 configured to operate the mechanical delivery system. For example, actuating the handle 130 through a first range of motion, the first jaw 102 and second jaw 110 hold the layers relative to one another. Actuating the handle further through a second range of motion ejects one of the elongated member 202 or collar 210 (e.g., by moving an ejection jaw 118), such that the elongated member 202 and collar 210 are coupled, thereby securing the layers therebetween. In some implementations, the delivery system of the fastener dispenser 100 may include a controlled delivery mechanism, which allows for precise deployment of the tissue fasteners 200. The mechanism is designed to prevent excessive force on the tissue by the fastener dispenser 100 and the fastener 200, which could potentially reduce ischemia and necrosis of the surrounding tissues. With this feature, the fastener dispenser can help ensure that the tissue is not damaged during the fastening process, which may lead to better patient outcomes and lower risks of complications. While this disclosure often refers to the two-stage delivery system as mechanical, it is contemplated that the delivery system could also be electromechanical.

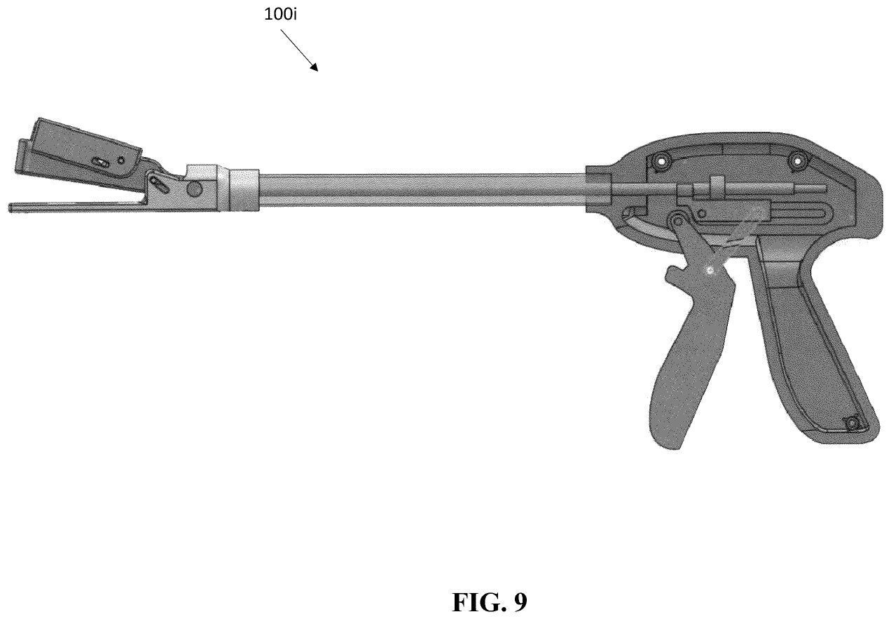

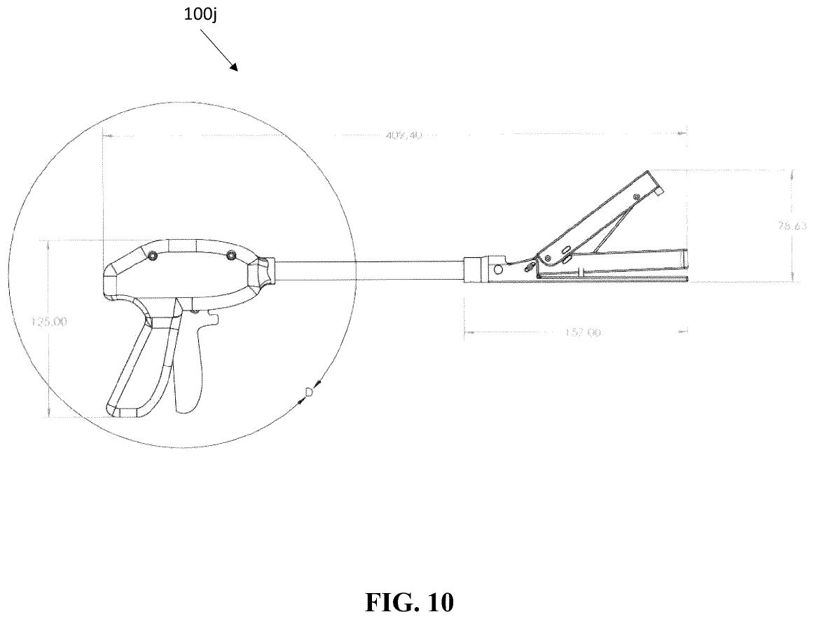

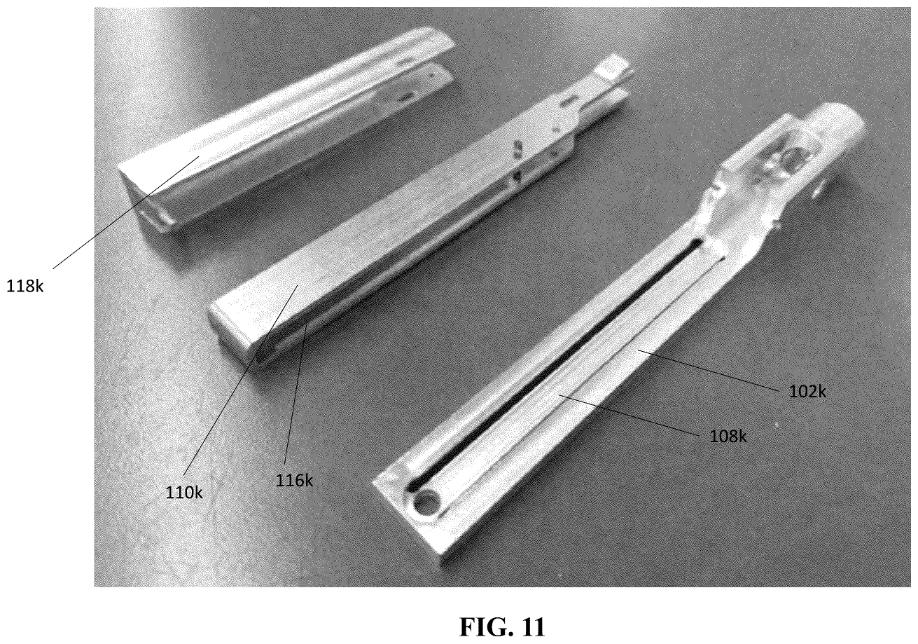

Additional exemplary fastener dispensers 100 (shown as 100i and 100j) are illustrated in FIGS. 9 and 10. As shown, for example, in FIG. 11, one component of the fastener may be stored in a first track 108 (shown as 108k) extending along a length of the first jaw 102 (shown as 102k), and a second component of the fastener may be stored in a second track 116 (shown as 116k) extending along the length of the second jaw 110 (shown as 110k).

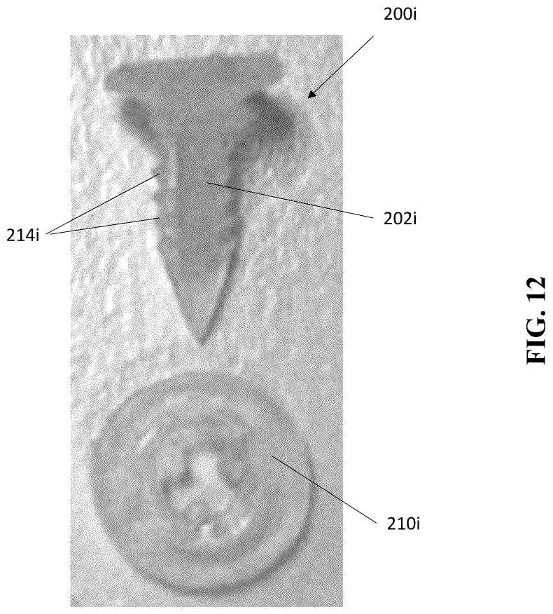

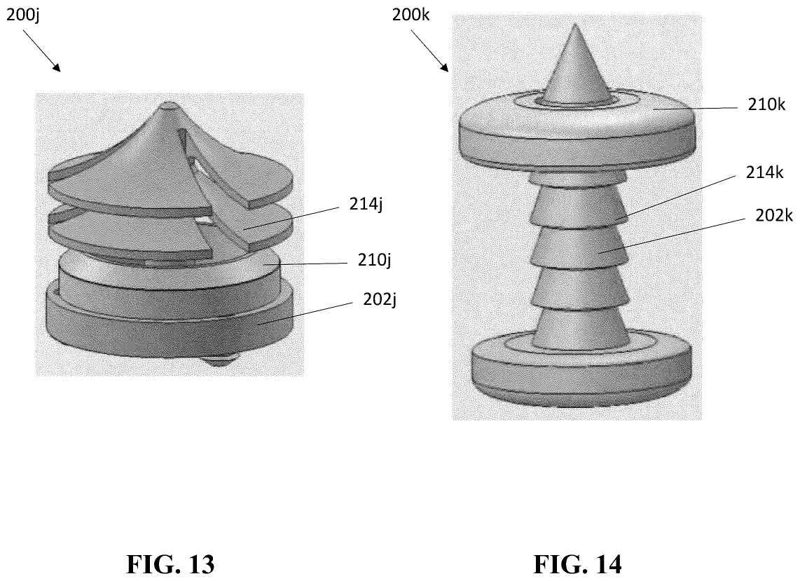

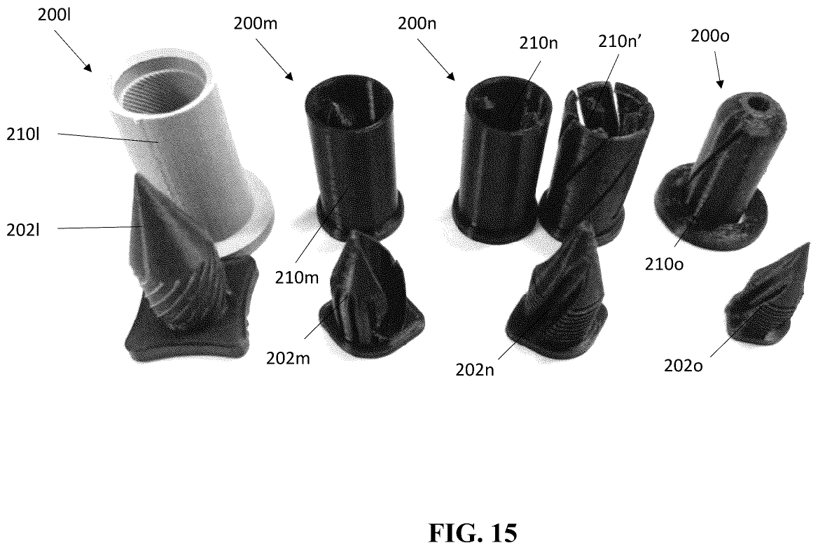

Example Fasteners. In various implementations, the fastener 200 includes an elongated member 202 (e.g., a penetrating tack) and a retention collar 210. In use, the fastener 200 can be deployed to fix (i.e., join) two or more soft layers. In some implementations, the elongated member 202 is tapered and includes a high-pitch spiral to aid in tissue penetration, and a series of engagement features 214 (e.g., ribs) to allow for variable thickness placements. In other words, as shown for example in FIGS. 12, 13, and 14, the engagement features 214 (shown as 214i-214k) may include a plurality of engagement features 214 extending along a length of the elongated member 202 such that the collar 210 (shown as 214i-214k) may be coupled to the elongated member 202 at any one or more of the engagement features 214. For example, when fixing relatively thin layers together, the collar 210 may engage with an engagement feature 214 disposed near the base end 204 of the elongated member 202. Conversely, when fixing relatively thick layers together, the collar 210 may engage with an engagement feature 214 disposed near the penetrating end 206 of the elongated member 202. In this way, the fastener 200 is able to accommodate variations in the thickness of the layers to be fixed together. Additional exemplary embodiments of the fastener 200 (shown as 200l-200o) are shown in FIG. 15.

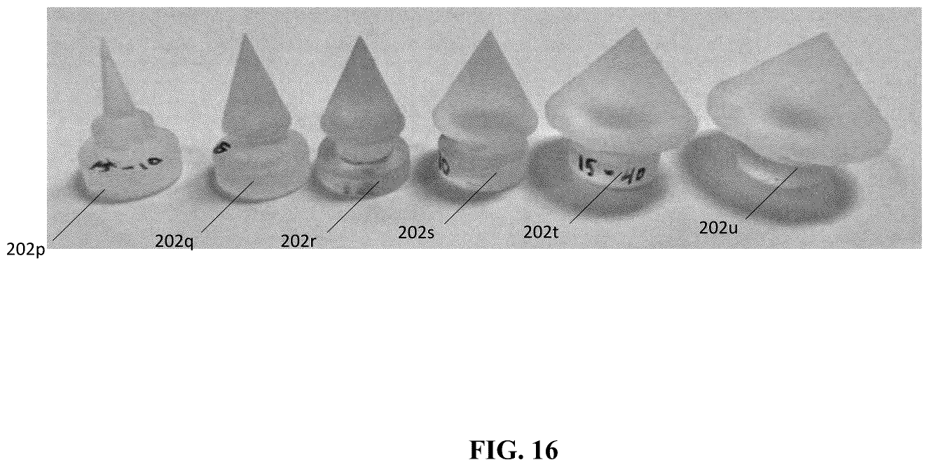

FIG. 16 show additional implementations of the elongated member 202 (shown as 202p-202u). In the illustrated implementation, the elongated member 202 is formed from a resin using SLA 3-Dimensional (“3D”) printing. In some implementations, one or both of the elongated member 202 and collar 210 are manufactured using SLA 3-dimensional printing with a durable resin. In some implementations, the fastener components, including the elongated member 202 and collar 210, are composed of resorbable material. However, in other implementations, non-resorbable materials may be used.

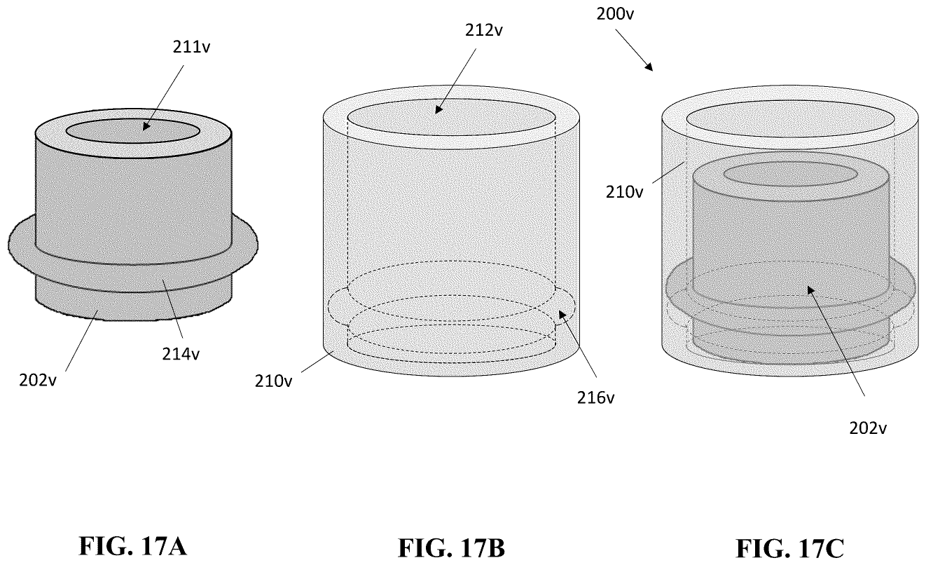

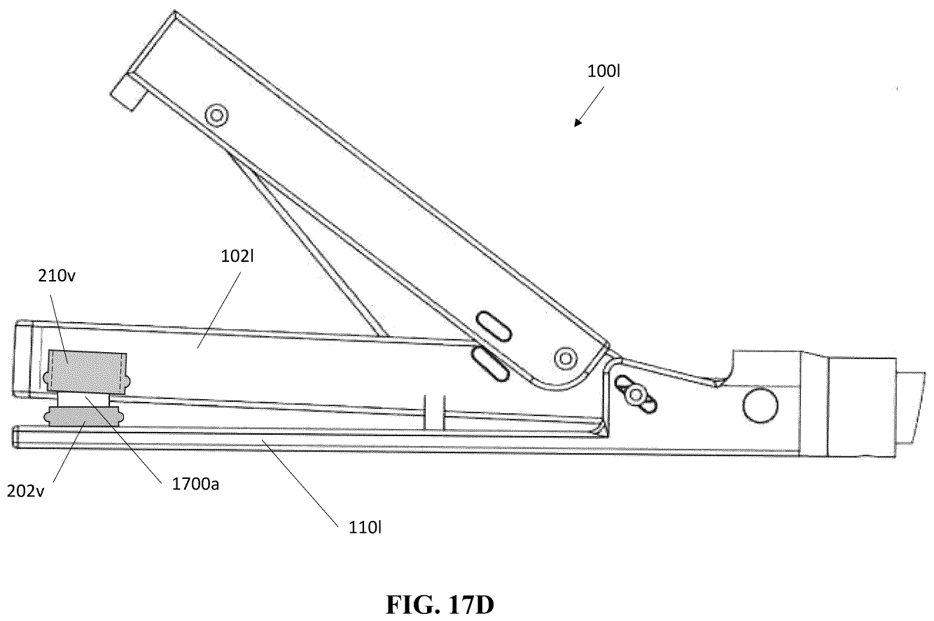

Referring generally to figures, FIGS. 17A-17C show yet another exemplary fastener 200 (shown as 200u), including an elongated member 202 (shown as 202v) and a collar 210 (shown as 210v). In the implementation illustrated in FIG. 17A, the elongated member 202 is formed as a tubular body and defines an opening 211 extending therethrough. Furthermore, the elongated member 202 includes a radially extending engagement feature 214 (shown as 214v). Specifically, FIG. 17A shows an elongated member 202 with a radially outwardly extending engagement feature 214 in the shape of an annular ring. The radially outwardly extending engagement feature 214 may extend all or partially around the elongated member 202 and may include various other geometries configured to facilitate one-way coupling of the elongated member 202 with the collar 210.

As shown in FIG. 17B, the collar 210 defines an opening 212 (shown as 212u) configured to receive the elongated member 202. As shown, the collar 210 further includes a radially extending engagement feature 216 configured to couple with the radially extending engagement feature 214 of the elongated member 202. Accordingly, the radially extending engagement feature 216 of the collar 210 may be radially inwardly extending or radially outwardly extending. In the illustrated implementation, the radially extending engagement feature 216 of the collar 210 is radially inwardly extending. FIG. 17C shows the tubular elongated member 202 and tubular collar 210 in the coupled configuration such that the engagement features 214 and 216 are engaged.

FIG. 17D shows an exemplary fastener dispenser 100 (shown as 100l). As shown, the second jaw 110 (shown as 110l) includes a guide member 1700 (shown as 1700a) extending toward the first jaw 102 (shown as 102l). The guide member 1700 is configured to align the elongated member 202 with the opening 212 of the collar 210.

### Example Surgical Method

In various implementations, the method for surgically fixing a first layer to a second layer includes fixing an implant mesh to patient tissue using an underlay technique. For example, in the underlay technique, the elongated member 202 may be positioned anteriorly to the implant mesh. The penetrating end 206 of the elongated member 202 penetrates through the implant mesh, fascia, and abdominal wall, the fastener collar 210 is coupled to the elongated member 202. Accordingly, the implant mesh, fascia, and abdominal wall may be fixed between the base end 204 of the elongated member 202 and the collar 210.

The surgical systems and methods provided herein offer several advantages. For example, use of the fastener dispenser 100 to dispense the fasteners 200 reduce surgical time, improves fixation the durability of the fixation, simplifies and standardizes the accompanying fixation procedure, and reduce physician fatigue commonly associated with large size hernia repair. As described herein, the disclosed systems and methods may be used in various other contexts. For example, the device could be used in inguinal hernia repairs or as a general tissue-to-tissue fixation system. Advantageously, the disclosed fastener 200 uses two components to perform fixation. This is advantageous in that no engagement geometries, such as screws or barbs, interface directly with the patient's tissues. Rather, any such features engage with the other components of the fastener.

The surgical fastening system provided herein is designed to be compatible with a wide range of surgical meshes. However, in some contexts of use, (e.g., fixing a surgical mesh to a patient's tissue), users may find it beneficial to make a small surgical adjustment by creating a central hole enabling the delivery tool's first jaw 102 to pass through below the surgical mesh. To minimize the risk of harming bowel tissues beneath the mesh, the first jaw 102 of the fastener dispenser may incorporate various geometries and features for the prevention of ischemia and necrosis, thereby reducing potential harm accompanying mesh insertion. By incorporating these protective measures, the fastener dispenser can help to ensure safer and more effective mesh implantation procedures. Furthermore, the surgical fastening system may be particularly suitable for use in conjunction with surgical meshes that include slotted mesh pockets on their posterior surface (e.g., Ventralex, Ventrio ST, etc.).

### Additional Example Fastener and Dispenser System

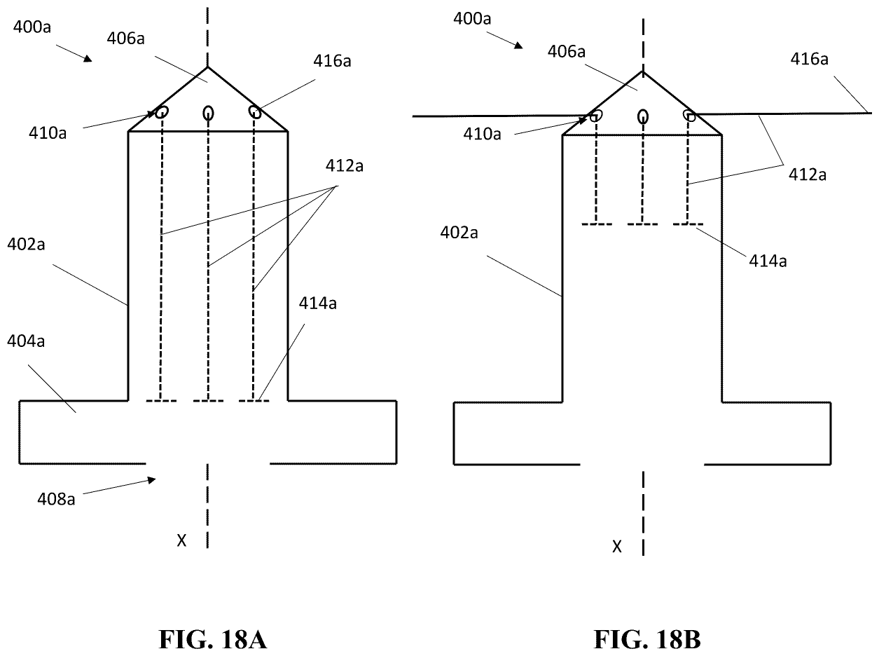

In some instances, conventional tissue fasteners may not form a strong coupling between two layers of tissue targeted for fixation. This can be due at least in part to poor penetration of the fastener within the targeted tissue and/or poor retention of the fastener within the targeted tissue once placed. Furthermore, in some instances, it may be desirable to secure a first layer to a second layer (e.g., fix a surgical mesh, such as a hernia mesh, to a patient's tissue or two layers of tissue to each other) via especially minimally invasive means. Accordingly, an additional implementation of the fastener is provided herein in FIGS. 18A-21C with a fastener delivered through a delivery cannula.

FIGS. 18A-18B, show an exemplary fastener 400 (shown as 400a) for securing a first layer to a second layer. As shown in the example of FIG. 18A, the fastener 400 includes an elongated member 402 (shown as 402a) that extends between a base end 404 (shown as 404a) and a penetrating end 406 (shown as 406a). The fastener 400 defines a longitudinal axis X and a fastener lumen 408 (shown as 408a) extending longitudinally through the elongated member 402 parallel with the longitudinal axis X. In the illustrated implementation, the fastener lumen 408 is open at the base end 404 and closed at the penetrating end 406. In some implementations, the penetrating end 406 tapers to a pointed tip that facilitates entry into the first and second layers (e.g., patient tissue and/or surgical mesh) As shown, the elongated member 402 defines a plurality of openings 410 (shown as 410a) that extend between the outer surface of the elongated member 402 to the fastener lumen 408. The plurality of openings 410 can be arranged along the tapered portion of the penetrating end 406, as shown, or be disposed along the longitudinally extending portion of the elongated member 402. In the illustrated implementation, the plurality of openings 410 are arranged circumferentially around the tapered region of the penetrating end 406 about the longitudinal axis X.

Although the illustrated plurality of openings 410 are ovoid, in further implementations, the plurality of openings 410 can be any shape, so long as the plurality of anchoring members 412 are able to move therethrough.

In the illustrated implementation, the fastener 400 has a length of about 2 cm as measured between the base end 404 to the penetrating end 406. This approximately corresponds to the width of the layers that the fastener 400 is configured to secure to each other. Accordingly, the dimensions of the fastener 400 can be varied to accommodate patients, tissues, and/or implanted layers of differing sizes. For example, the length of the fastener 400 can range from about 0.5 cm to about 3.0 cm. For example, the length of the fastener 400 can range from about 1.0 cm to about 2.0 cm.

A plurality of longitudinally extending anchoring members 412 (shown as 412a) extend longitudinally within the fastener lumen 408. As shown, each anchoring member 412 includes a proximal end 414 (show as 414a) and a distal end 416 (shown as 416a). In the illustrated implementation, the anchoring members 412 are arranged circumferentially about the longitudinal axis X.

In the illustrated implementation, each anchoring member 412 is formed from a deformable polymeric material. However, in further implementations, the plurality of anchoring members 412 can be formed from any material that allows the anchoring member 412 to bend. Additionally, in some implementations, the plurality of anchoring members 412 can be formed from a biodegradable material. In specific examples, the character of the biodegradable material used to form the plurality of anchoring members 412 can be controlled to modulate degradation time.

Furthermore, each anchoring member 412 in the illustrated implementation has a rectangular cross-section. The relative cross-sectional dimensions of each anchoring member 412 can be controlled to adjust the stiffness of the respective anchoring member 412.

As further described herein, the plurality of anchoring members 412 are movable from an undeployed configuration to a deployed configuration. In the undeployed configuration, the plurality of anchoring members 412 are disposed entirely within the fastener lumen 408. As showed in the illustrated implementation, when in the undeployed configuration, the plurality of anchoring members 412 can extend parallel to the longitudinal axis X of the fastener 400. In the deployed configuration, at least a portion of two or more of the anchoring members 412 move radially outward and extend through a respective opening 410. Specifically, the plurality of anchoring members 412 are configured to move from the undeployed configuration to the deployed configuration upon axial translation of at least the proximal end of respective anchoring members 412 toward the penetrating end 406 of the fastener 400. Advantageously, the plurality of anchoring members 412 increase the pull out force required to withdrawal the fastener 400, thereby improving retention of the fastener 400 within the first and second layers and improving the secured coupling formed between the first layer and the second layer by the fastener 400. In various implementations, each anchoring member 412 can extend radially outward as a distance ranging from about 0.5 cm to about 2.0 cm as measured between the outer surface of the elongated member 402 and the distal end 416. In the illustrated implementation, each anchoring member 412 extends radially outward a distance of about 1.0 cm.

FIG. 18B shows the exemplary fastener 400 with the plurality of anchoring members 412 in the deployed configuration. As shown, advancement of the plurality of anchoring members 412 axially toward the penetrating end 406 such that the distal ends 416 of each respective anchoring member 412 is directed through a respective opening 410 and radially outward. Although the illustrated implementation shows each anchoring member extending radially outward at a 90 degrees angle relative to the longitudinal axis, in further implementations, each anchoring member can extend radially outward so as to form an acute angle with respect to the longitudinal axis. Advantageously, this can further improve retention of the fastener 400 within the first and second layers by increasing the withdrawal force required to dislodge the fastener 400. For example, the acute angle can range from about 89 degrees to about 15 degrees. For example, the acute angle can range from about 60 degrees to about 30 degrees. For example, the acute angle can be about 45 degrees.

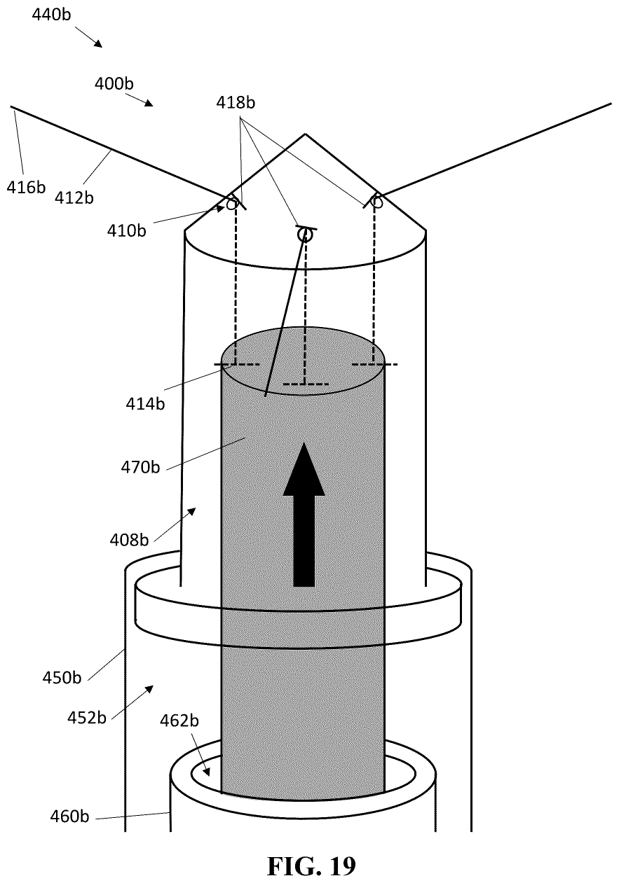

FIG. 19 shows another implementation of the fastener 400 (shown as 400b) in a part of a surgical fastening system 440 (shown as 440b). As shown, the illustrated fastener 400 further includes a plurality of inner shelves 418 disposed adjacent respective openings 410. The plurality of inner shelves 418 can be protrusions formed within the fastener lumen 408 that are angled to direct the distal end 416 of the plurality of anchoring members 412 radially outward as the plurality of anchoring members 412 advances toward the penetrating end 406 when transitioning from the undeployed configuration to the deployed configuration. Advantageously, the plurality of inner shelves 418 can divert the trajectory of the plurality of anchoring members 412 away from the longitudinal axis X and radially outward into the first or second layer (e.g., patient tissue).

Furthermore, as shown in FIG. 19, the surgical fastening system 440 includes a delivery cannula 450 (shown as 450b) having a central lumen 452 (shown as 452b) in which the fastener 400 is disposed. Disposed within the central lumen 452 of the delivery cannula 450 is a tubular fastener driver 460 (shown as 460b) having a central lumen 462 (shown as 462b). As further described herein, the fastener driver 460 is configured to move axially within the central lumen 452 of the delivery cannula 450 to apply a distally-directed axial force on the base end 404 of the fastener 400 to advance the fastener 400 out of the delivery cannula 450 and into the first and second layers.

Disposed within the central lumen 462 of the fastener driver 460 is an anchor driver 470 (shown as 470b) in the form of a push-rod. As shown by the arrow in FIG. 19, the anchor driver 470 is configured to translate axially parallel to the longitudinal axis X within the central lumen 452 of the delivery cannula 450 and engage the proximal ends 414 of respective anchoring members 412, thereby moving respective distal ends 416 of the plurality of anchoring members 412 toward respective inner shelves 418 and thus diverting the plurality of anchoring members 412 radially outward toward through respective openings 410.

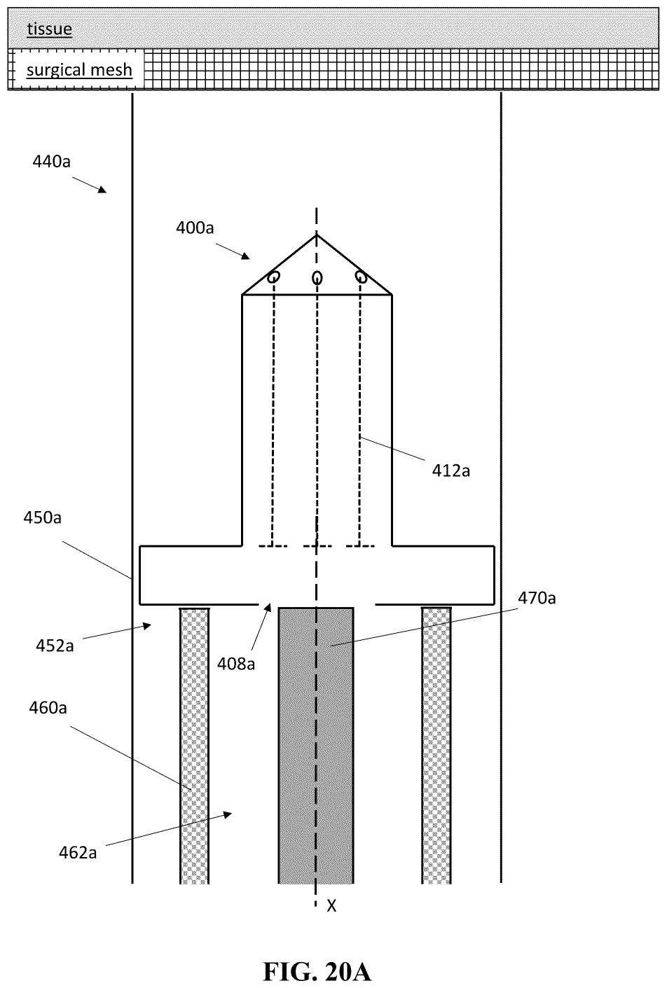

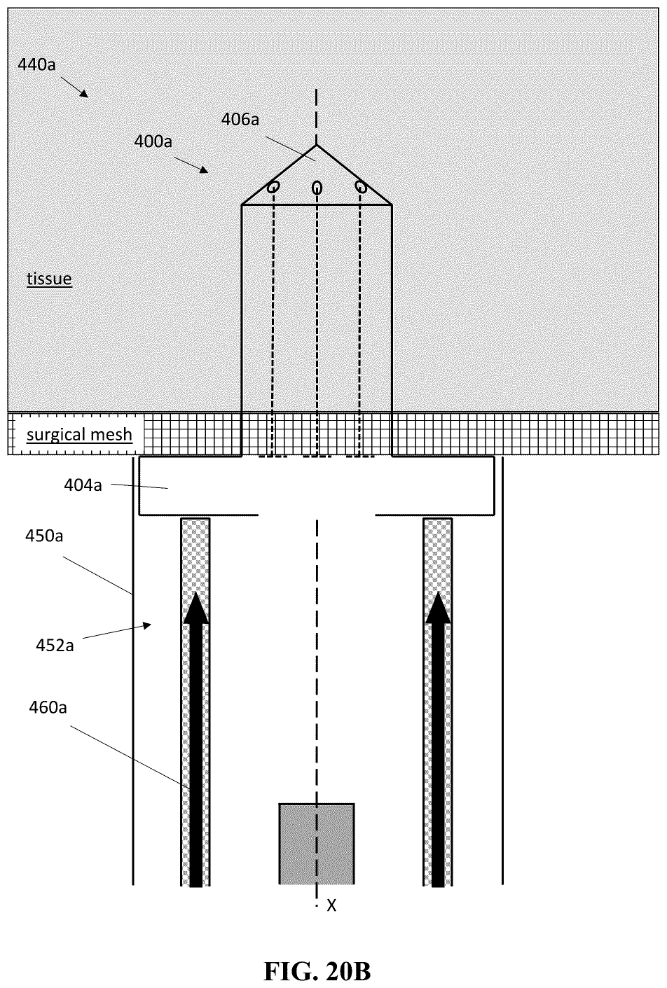

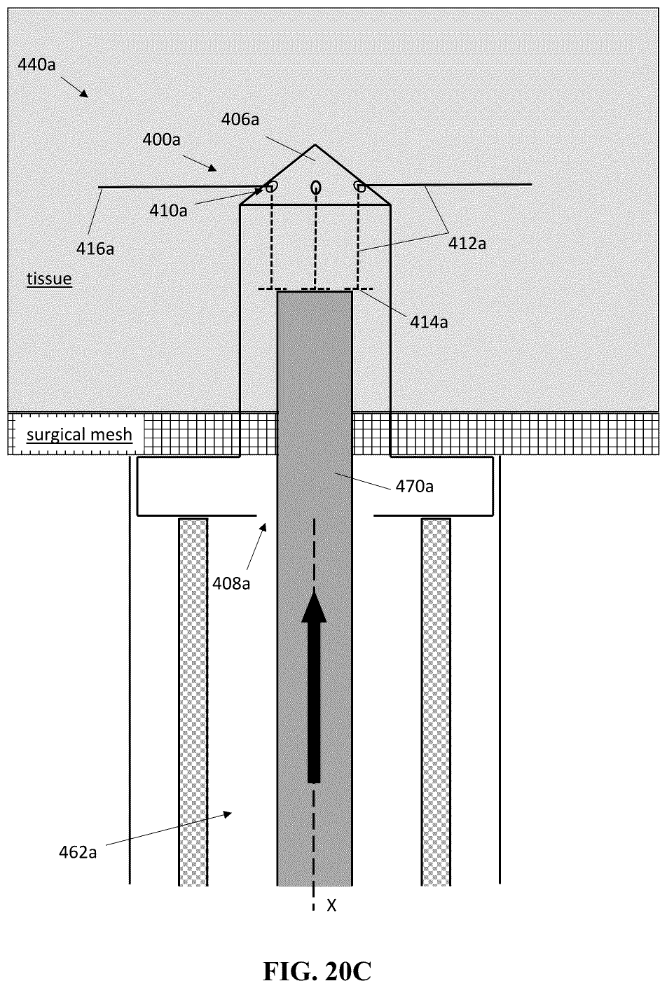

Referring now to FIGS. 20A-20C, an exemplary surgical fastening system 440 (shown as 440a) is shown in use in conjunction with the exemplary fastener 400 (shown as 400a) along with the delivery system including the exemplary delivery cannula 450 and exemplary anchor driver 470. Specifically, FIG. 20A shows the fastener 400, the fastener driver 460, and the anchor driver 470 disposed within the central lumen 452 of the delivery cannula 450. As shown, the delivery cannula 450 is positioned adjacent the first and second layers such that distal end of the delivery cannula 450 contacts whichever of the first layer or second layer is nearest the delivery cannula 450. In the illustrated example, the first layer is a surgical mesh (e.g., hernia mesh). However, in other implementations, the first and second layer may both be layers of patient tissue. While the fastener 400 remains disposed within the central lumen 452 of the delivery cannula 450, the plurality of anchoring members 412 are retained fully within the fastener lumen 408.

When the delivery cannula 450 is aligned with the desired location for securing the two layers together, the fastener driver 460 is advanced axially in a distal direction, as shown by the arrows in FIG. 20B, so that the fastener driver 460 contacts the base end 404 of the base end 404. The fastener driver 460 then pushes the penetrating end 406 of the fastener 400 distally parallel to the longitudinal axis X and into the first layer and second layer. In the illustrated implementation, the fastener 400 is advanced distally until the base end 404 abuts the layer nearest the delivery cannula 450. In the particular implementation illustrated, the fastener 400 is advanced through a first layer of surgical mesh and further advanced into a second layer of patient tissue.

Once the fastener 400 is positioned so as to extend through all or part of the first and second layers, the anchor driver 470 is advanced axially in a distal direction, as shown by the arrow in FIG. 20C, so that the anchor driver 470 contacts the respective proximal ends 414 of the plurality of anchoring members 412. As shown, this pushes the respective proximal ends of the plurality of anchoring members 412 distally toward the penetrating end 406 and directs the respective distal end 416 of the plurality of anchoring members 412 radially outward through the plurality of openings 410 and into the first or second layer (e.g., patient's tissue), thereby holding the fastener 400 within the first or second layer and securing the first layer and second layer to each other. Once the fastener 400 is place, the remainder of the surgical fastening system 440 including the delivery cannula 450, fastener driver 460, and anchor driver 470 can be withdrawn.

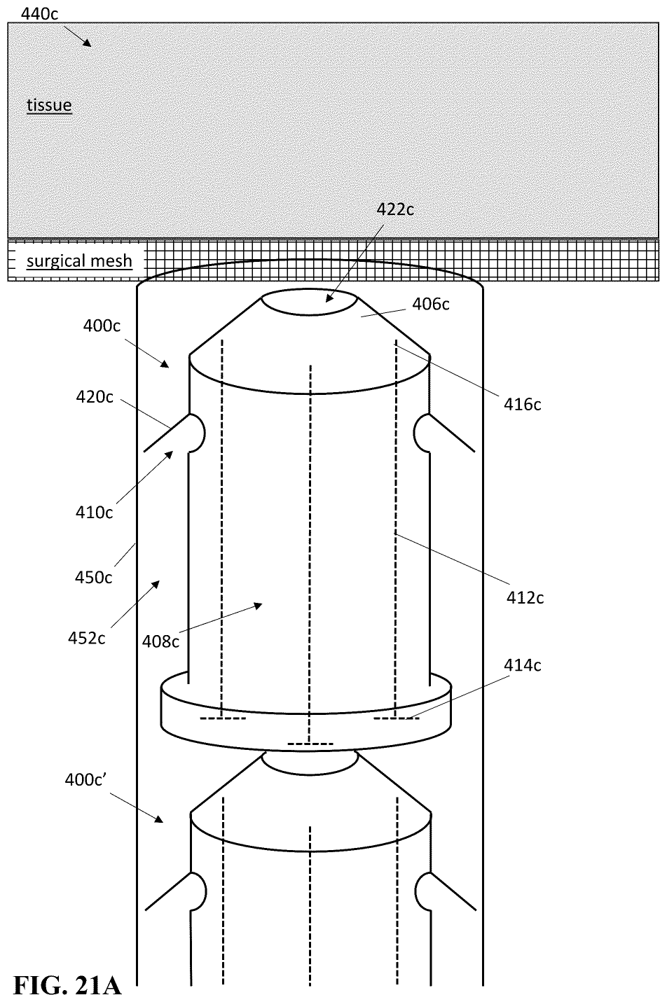

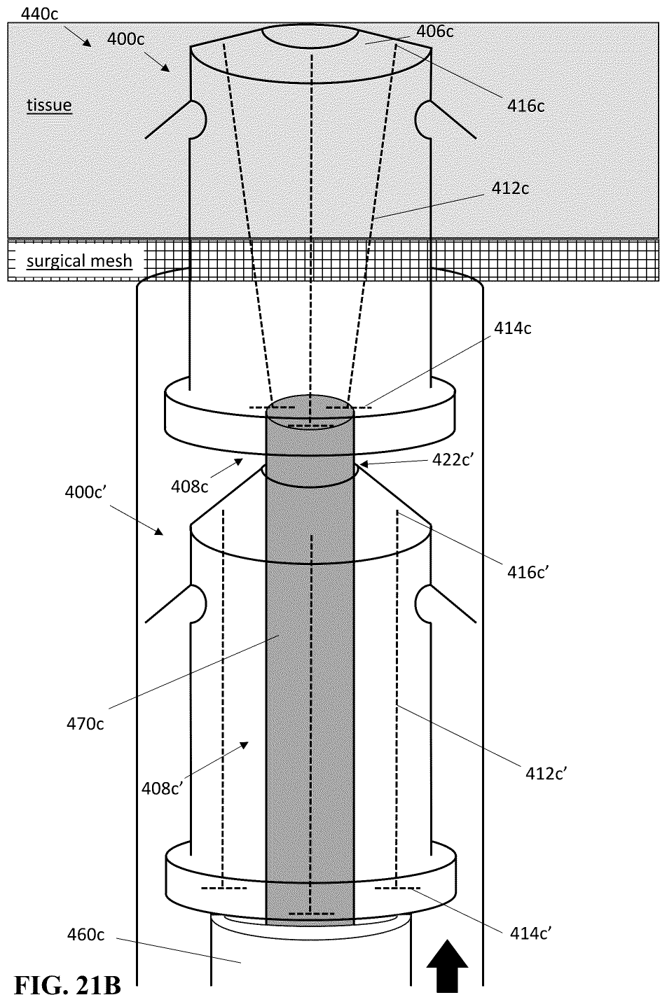

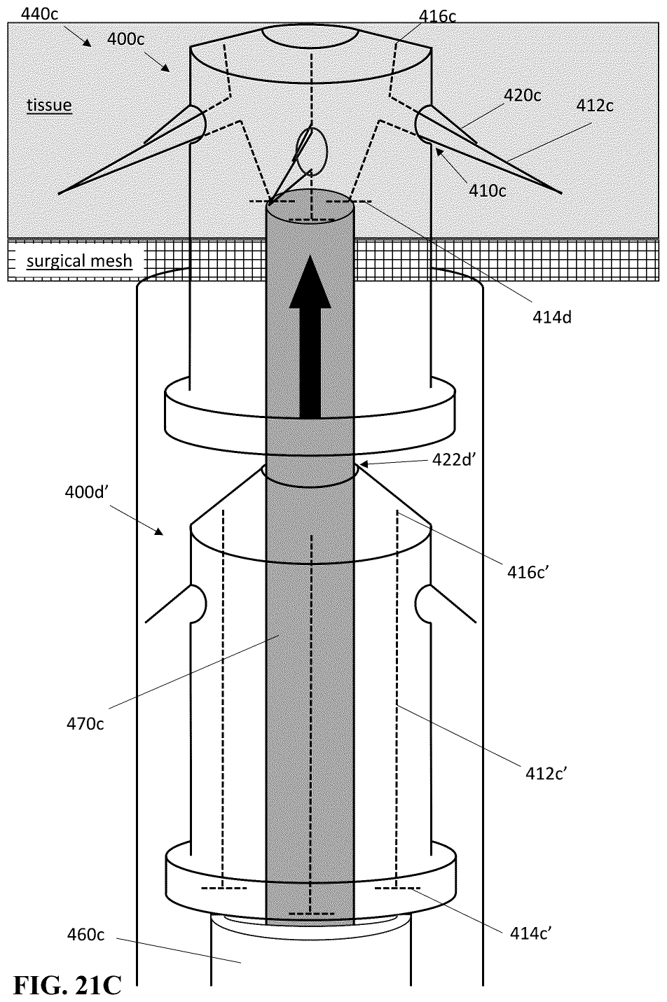

Referring now to FIGS. 21A-21C, another exemplary surgical fastening system 440 (shown as 440c) is shown in use in conjunction with the exemplary fastener 400 along with the delivery system including exemplary delivery cannula 450 and exemplary anchor driver 470. The surgical fastening system 440 shown in FIGS. 21A-21C is similar to the surgical fastening system 440 described with reference to FIGS. 20A-20C. However, the fastener 400 in this exemplary implementation (shown as 400c) includes a plurality of outer shelves 420 disposed adjacent respective openings 410. The plurality of outer shelves 420 can be protrusions formed along the outer surface of the elongated member 402 to a portion of each anchoring member 412 radially outward when transitioning from the undeployed configuration to the deployed configuration. Further still, as shown in the illustrated implementation, the plurality of outer shelves 420 are angled toward the base end 404 so as to direct the portion of each anchoring member 412 angularly toward the base end 404 so as to form an acute angle with respect to the longitudinal axis X. Advantageously, the plurality of inner shelves 418 can divert the trajectory of the plurality of anchoring members 412 away from the longitudinal axis X and radially outward into the first or second layer (e.g., patient tissue). Additionally, FIG. 21A shows that an additional fastener 400 (shown as 400c′) can be disposed within the delivery cannula 450 proximal to the fastener 400. In fact, it is contemplated herein that a series of fasteners fastener 400 can be contained within the delivery cannula 450 and configured to be deployed serially at various locations across the first and second layers targeted for securement.

As shown in previous implementations, the portion of each respective anchoring member that extends radially outward in the deployed configuration can be the distal end 416 of the respective anchoring member. Such a configuration is contemplated in combination with the plurality of outer shelves 420 as described above. However, in the illustrated implementation shown in FIGS. 2A-21C, the distal end 416 is fixed to an inner surface of the fastener lumen 408 at the penetrating end 406 of the fastener 400. As shown in FIG. 21B, in such implementations, the penetrating end penetrating end 406 of the fastener 400 is configured to collapse or depress upon engagement with the first and/or second layer. Because the distal end 416 are fixed, it is contemplated herein that such collapse of the penetrating end 406 urges the respective proximal ends 414 of the plurality of anchoring members 412 radially inward toward the longitudinal axis X.

Additionally, the fastener 400 defines a distal opening 422 (shown as 422c) in the penetrating end 406 such that the fastener lumen 408 extends fully through the fastener 400 between the base end 404 and the penetrating end 406.

As shown in FIG. 21B, the fastener 400 (shown as 400c) and one or more additional fasteners 400 (shown as 400c′) can be arranged end-to-end within the delivery cannula 450. As previously described, the fastener driver 460 translates distally within the central lumen 452 and engages the base end 404 of the fastener 400 (here, the additional fastener 400c′) to urge the fastener 400 (or series of fasteners 400) distally along the longitudinal axis X.

As further shown in FIG. 21B, the anchor driver 470 configured to move axially through the fastener lumens 408 of the one or more fasteners 400 disposed in the delivery cannula 450. For example, as shown, the anchor driver 470 extends entirely through the additional fastener 400 (shown as 400c′). In the illustrated implementation, this is possible because the plurality of anchoring members 412 offset from the longitudinal axis X a distance at least equal to the radius of the anchor driver 470. Thus, the anchor driver 470 passes between respective anchoring members 412. However, when the penetrating end 406 collapses and urges the respective proximal ends 414 of the plurality of anchoring members 412 radially inward toward the longitudinal axis X, as previously discussed, the respective proximal ends 414 are brought into alignment with the anchor driver 470 such that distal movement of the anchor driver 470 will push the respective proximal ends 414 distally. Advantageously, this arrangement enables several fasteners 400 to be stacked end-to-end within the delivery cannula 450 while facilitating deployment of the plurality of anchoring members 412 in only the distal-most fastener 400.

Referring now to FIG. 21C, distal advancement of the anchor driver 470 pushes the respective proximal ends 414 distally, moving the plurality of anchoring members 412 from the undeployed configuration into the deployed configuration. However, because the respective distal ends 416 of the plurality of anchoring members 412 are fixed to an inner surface of the fastener lumen 408 at the penetrating end 406 of the fastener 400, a portion of the respective anchoring members 412 disposed between the distal end 416 end and distal end 416 is urged through the respective openings 410 and radially outward into the first or second layer (e.g., patient tissue). As shown, the portion of the respective anchoring members 412 directed radially outward through the respective openings 410 is diverted by the plurality of outer shelves 420 so as to extend at an acute angle with respect to the longitudinal axis X. Accordingly, these portions of the respective anchoring members 412 can have any of the attributes of the radially extending distal end 416 described with respect to previous implementations. In this way, the fastener 400 can effectively anchor within the first and/or second layer and thus secure the first layer and second layer to each other. Once the fastener 400 is place, the remainder of the surgical fastening system 440 including the delivery cannula 450, fastener driver 460, and anchor driver 470 can be withdrawn.

Although some features of the fasteners 400 and surgical fastening systems 440 are described with respect to a single implementation (e.g., distal opening 422 and outer shelves 420 described in an embodiment having distal ends 416 fixed within the fastener lumen 408), it is contemplated herein that the attributes of some implementations are transferrable to other implementations. For example, the implementation in which the distal end 416 are moved radially outward through the plurality of openings 410 away from the longitudinal axis can also include a distal opening 422 and outer shelves 420.

The surgical fastening system 440 provided herein may be used in various minimally invasive and open surgical procedures. In various use cases provided herein, the surgical fastening system is employed to secure a surgical mesh to a target tissue in the course of surgical hernia repair, such as the repair of ventral and incisional hernias. In such uses, the surgical mesh is a hernia mesh, and the target tissue corresponds to a hernia defect site. However, the surgical fastening system may be utilized to secure other soft implants to other tissues or certain layers of tissue to other layers of tissue in the course of other surgical procedures. In some implementations, the implant is formed from an absorbable material, such as synthetic polyester copolymer derived from lactic and glycolic acid. In other implementations, the implant may be nonabsorbable.

### Configuration of Certain Implementations

The construction and arrangement of the systems and methods as shown in the various implementations, are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

1. A surgical fastening system for fixing a first layer to a second layer, the system comprising:
a fastener dispenser comprising:
a first jaw comprising a proximal end and a distal end, the first jaw defining a first jaw track therein extending between the proximal end and the distal end;
a second jaw comprising a proximal end and a distal end, the second jaw defining a second jaw track therein extending between the proximal end and the distal end; and
a fastener comprising an elongated member and a collar, the elongated member spanning the combined thickness of the first layer and the second layer, wherein the elongated member is stored within the second jaw track and the collar is stored within the first jaw track;
wherein the first jaw is movably coupled to the second jaw; and
wherein the fastener dispenser is movable from an unclamped configuration, in which the first jaw and the second jaw have a relative spacing on opposite sides of the first layer and the second layer, to a clamped configuration, in which the second jaw is moved relative to the first jaw to reduce the relative spacing between the first jaw and the second jaw, thereby holding the first layer relative to the second layer.)

2. The system of claim 1, wherein one of the first or second jaws further comprises an ejection jaw movably coupled to a counterpart one of the first or second jaws, the ejection jaw comprising a protrusion configured to extend toward the track of the one of the first or second jaws to which the ejection jaw is coupled;
wherein moving the ejection jaw toward the one of the first or second jaws engages the protrusion against one of the elongated member or the collar stored in the track of the one of the first or second jaws, thereby urging the one of the elongated member or the collar toward the other of the elongated member or the collar to couple the elongated member and the collar.

3. The system of claim 2, wherein an ejection jaw biasing member urges the ejection jaw toward the one of the first or second jaws to which the ejection jaw is coupled.

4. The system of claim 1, wherein the elongated member extends between a base end and a penetrating end and defines an elongated member central axis.

5. The system of claim 4, wherein the elongated member is stored within the second jaw track such that the elongated member central axis is parallel with or forms an acute angle with an extension axis of the second jaw track.

6. The system of claim 5, wherein the second jaw further comprises a pivoting feature configured to pivot the elongated member when the elongated member is dispensed such that the penetrating end of the elongated member extends toward the collar.

7. The system of claim 4, wherein the system further comprises a cutting blade disposed adjacent the first jaw, the cutting blade configured to remove a portion of the penetrating end of the elongated member on a side of the collar opposite the base end of the elongated member after the elongated member is coupled to the collar.)

8. The system of claim 1, wherein the system further comprises a collar cartridge configured to store and dispense the collar, wherein the collar is one of a plurality of collars stored in the collar cartridge, and wherein a first biasing member urges the collar cartridge toward the distal end of the first jaw

9. (canceled)

10. The system of claim 8, wherein the system further comprises an elongated member cartridge configured to store and dispense the elongated member, wherein the elongated member is one of a plurality of elongated members stored in the elongated member cartridge, and wherein a second biasing member urges the elongated member cartridge toward the distal end of the second jaw.

11. (canceled)

12. The system of claim 10, wherein the first jaw track is configured to receive the collar cartridge, and the second jaw track is configured to receive the elongated member cartridge.

13.-14. (canceled)

15. The system of claim 1, wherein the elongated member is formed integrally with the second jaw and extends toward the first jaw, wherein the system further comprises a dispensing blade configured to remove the elongated member from the second jaw.

16. The system of claim 2, wherein the system further comprises a housing, the housing comprising a first actuator movable between a first unengaged position and a first engaged position, wherein movement between the first unengaged position and the first engaged position moves the fastener dispenser between the unclamped configuration to the clamped configuration, respectively.

17. The system of claim 16, wherein the housing further comprises a second actuator movable between a second unengaged position and a second engaged position, wherein movement between the second unengaged position and the second engaged position moves the ejection jaw relative to one of the first or second jaws to urge one of the elongated member or the collar toward the other of the elongated member or the collar to couple the elongated member and the collar.

18. The system of claim 17, wherein the system further comprises a handle configured to move the first actuator between the first unengaged position and the first engaged position.

19. The system of claim 18, wherein the handle is further configured to move the second actuator between the second unengaged position and the second engaged position.

20. The system of claim 17, wherein the system further comprises a hollow shaft extending from the housing to the fastener dispenser.

21. The system of claim 20, wherein the first jaw is rotationally fixed to the hollow shaft.

22. The system of claim 20, wherein the fastener dispenser is detachably coupled to the hollow shaft for disposal or replacement.

23. The system of claim 1, wherein the first layer is a surgical mesh implant, and the second layer is a patient's tissue.

24. The system of claim 23, wherein the surgical mesh implant is a hernia mesh.

25. The system of claim 1, wherein both the first layer and the second layer are a patient's tissue.

26. A surgical fastener for fixing a first layer to a second layer, the fastener comprising:
an elongated member extending between a base end and a penetrating end and defining an elongated member central axis; and
a collar defining an opening configured to receive the penetrating end of the elongated member;
wherein the elongated member spans the combined thickness of the first layer and the second layer;
wherein the elongated member is configured to engage with the opening of collar, thereby coupling the elongated member and the collar; and
wherein the fastener operates in a system further comprising a fastener dispenser, the fastener dispenser comprising:
a first jaw comprising a proximal end and a distal end, the first jaw defining a first jaw track therein extending between the proximal end and the distal end; and
a second jaw comprising a proximal end and a distal end, the second jaw defining a second jaw track therein extending between the proximal end and the distal end;
wherein the proximal end of the first jaw is pivotably coupled to the proximal end of the second jaw; and
wherein the elongated member is stored within the second jaw track and the collar is stored within the first jaw track.

27.-30. (canceled)

31. A method for surgically fixing a first layer to a second layer, the method comprising:
(1) providing a fastener dispenser comprising:
a first jaw having a proximal end and a distal end, the first jaw defining a first jaw track therein extending between the proximal end and the distal end;
a second jaw having a proximal end and a distal end, the second jaw defining a second jaw track therein extending between the proximal end and the distal end;
(2) providing a fastener comprising:
an elongated member and a collar, the elongated member spanning the combined thickness of the first layer and the second layer,
wherein the elongated member extends between a base end and a penetrating end and defines an elongated member central axis;
wherein the collar defines an opening configured to receive the penetrating end of the elongated member; and
wherein the elongated member is stored within the second jaw track and the collar is stored within the first jaw track;
(3) positioning the fastener dispenser so that the first layer and the second layer are disposed between the first jaw and the second jaw;
(4) moving the fastener dispenser from an unclamped configuration, in which the first jaw and the second jaw have a relative spacing on opposite sides of the first layer and the second layer, to a clamped configuration, in which the second jaw is pivoted relative to the first jaw to reduce the relative spacing between the first jaw and the second jaw, thereby holding the first layer relative to the second layer; and
(5) coupling the elongated member with the collar, thereby clamping the first layer and the second layer between the base end of the elongated member and the collar, thereby fixing the first layer relative to the second layer.

32.-55. (canceled)
